(19) United States
(12) Patent Application Publication
(10) Pub. No.: US 2020/0025762 A1
(43) Pub. Date: Jan. 23, 2020

(54) NON-TARGET SITE RESISTANCE

(71) Applicant: UNIVERSITY OF NEWCASTLE UPON TYNE, Tyne and Wear (GB)

(72) Inventors: Robert Edwards, Tyne and Wear (GB); Catherine Tetard-Jones, Tyne and Wear (GB); Nawaporn Onkokesung, Tyne and Wear (GB); Melissa Catherine Brazier-Hicks, Tyne and Wear (GB); Alina Viviana Goldberg Cavalleri, Tyne and Wear (GB)

(73) Assignee: UNIVERSITY OF NEWCASTLE UPON TYNE, Tyne and Wear (GB)

(21) Appl. No.: 16/481,213

(22) PCT Filed: Jan. 25, 2018
(86) PCT No.: PCT/GB2018/050206

§ 371 Date: Jul. 26, 2019

(30) Foreign Application Priority Data
Jan. 27, 2017 (GB) 1701349.1

(51) Int. Cl. G01N 33/573; C12Q 1/6895

(52) U.S. Cl. CPC G01N 33/573; C12Q 1/6895; C12Q 2600/158; G01N 2560/00; C12Q 2600/13; G01N 2333/415

(57) ABSTRACT

The present invention provides novel biomarkers for non-target-site resistance (NTSR) in wild grass, with corresponding methods of identifying NTSR. Corresponding kits and assay devices are also provided.

The present invention provides novel biomarkers for non-target-site resistance (NTSR) in wild grass, with corresponding methods of identifying NTSR. Corresponding kits and assay devices are also provided.

### BACKGROUND

Herbicides are widely used in modern agriculture as a mechanism for controlling unwanted plant growth. Herbicides function by binding to a plant target site, where they act to disrupt a particular plant process or function. Typically, herbicides bind to and inactivate an enzyme that is essential to the plant's normal physiological activity (e.g. an enzyme involved in protein or fatty acid synthesis).

The development of herbicide resistance is a significant and growing concern. In the UK, herbicide resistance in wild grasses such as black-grass (Alopecurus myosuroides) has led to loss of chemical control in large parts of the country, with substantial annual losses of wheat yield.

Herbicide resistance may be classified as target-site resistance or non-target-site resistance, depending on the underlying cause. Target-site resistance (TSR) arises through mutation in a target site of herbicide action, which adversely affects herbicide binding without disrupting the targeted process or function of the plant. By way of example, a mutation may occur in a gene encoding a target enzyme, which alters the enzyme binding site (but not the enzyme's normal physiological function). Such mutations may prevent the herbicide from binding to and inactivating the target enzyme, without affecting enzyme function. TSR is a well-characterised resistance mechanism and the mutations creating resistance against each herbicide mode of action have been well catalogued in many weed species (reviewed by Powles and Yu 2010).

The second type of resistance arises at sites other than those targeted directly by herbicides and as such is termed non-target site resistance (NTSR). The mechanisms giving rise to NTSR are complex and only partially understood. In the case of black-grass, the first reported NTSR population was identified near the village of Peldon in Essex in the 1980's, at a site associated with intense and long-term herbicide usage (Hall et al., 1997; Moss, 1990). NTSR in black-grass has since steadily established itself in areas where winter wheat is a dominant crop and is now widespread in populations across the UK and Benelux countries. NTSR arises through the avoidance of irreversible cellular damage brought about through detoxification, exclusion, sequestration, suppression of down-stream toxicity or a combination of these cytoprotective responses following chemical exposure. NTSR may therefore result from one or more mechanisms, including metabolic based resistance (e.g. NTSR based upon enhanced herbicide detoxification or enhanced tolerance of stress caused by herbicide toxicity), reduced herbicide penetration through leaf tissue, target enzyme overproduction, or protection against oxidative stress and other downstream cytotoxic events caused by chemical injury.

The specific mechanism underlying the NTSR phenotype of a plant population may result in a general defence to herbicides (irrespective of the herbicide's specific mode of action). By way of example, NTSR based upon metabolic based resistance typically provides a general defence to herbicides and as such the NTSR population is typically resistant to multiple classes of herbicides. Alternatively, the underlying mechanism of the NTSR phenotype may result in a more specific resistance to one herbicide (or a class of herbicides with a common mode of action).

Identifying whether or not a weed population has NTSR (and characterising the mechanisms underlying the NTSR phenotype) is of fundamental importance as it influences the control measures to be taken, be they chemical or cultural. Accordingly, there is a need to identify novel biomarkers of NTSR.

### BRIEF SUMMARY OF THE DISCLOSURE

The inventors have conducted comparative transcriptomic and proteomic analysis of NTSR and herbicide susceptible (HS) black-grass populations. Both field collected populations and populations that had been experimentally selected for herbicide resistance were analysed. Several transcripts and corresponding proteins had increased abundance in NTSR black-grass compared to HS black-grass.

To explore the origins of NTSR, the inventors analysed the proteome of HS blackgrass exposed to a range of biotic (insect feeding, plant-microbe interaction) and abiotic stresses (N-limitation, osmotic, heat), including herbicide safening. It was found that the vast majority of changes in protein abundance associated with NTSR were distinct from the changes in protein abundance observed in response to stress. Proteins with a change in abundance in NTSR samples therefore serve as bona fide biomarkers of NTSR.

The data was further analysed to identify proteins with at least a 1.5 fold increased abundance in at least one of the tested NTSR populations compared to HS. As expected, the glutathione transferase AmGSTF1 was found to be increased in each of the NTSR populations. This is in line with previous findings, where AmGSTF1 was identified as a functional biomarker for NTSR in black-grass (Cummins et al., 1999; Cummins et al., 2013). However, a further seven previously undescribed proteins were identified with increased abundance in three NTSR populations (peldon, oxford and pendimethalin) that were resistant to multiple herbicides. In contrast, these proteins were not enhanced in an NTSR population that was resistant to fenoxaprop only. The seven novel biomarkers therefore provide additional valuable information on the type of NTSR present in the tested plant populations.

The invention is based on the finding that GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”) are each useful biomarkers for NTSR in wild grass, wherein the NTSR is based upon metabolic based resistance conferring resistance to multiple herbicides.

The invention also provides corresponding methods of identifying NTSR, wherein the NTSR is based upon metabolic based resistance conferring resistance to multiple herbicides. Corresponding kits and assay devices are also provided.

In one aspect, the invention provides the use of at least one of:
i. GSTU2;
ii. D-3-phosphoglycerate dehydrogenase 1;
iii. 12-oxophytodienoate reductase 1;
iv. GSTF2;
v. NADPH:quinone oxidoreductase 1;
vi. NAD-dependent epimerase/dehydratase;
vii. stem-specific protein TSJT1;

as a biomarker for non-target-site herbicide resistance (NTSR) in wild grass, wherein the NTSR is based upon metabolic based resistance.

Optionally, the biomarker is protein or mRNA.

Optionally, the wild grass is selected from the group consisting of black grass, rye grass, wild oat and bent grass.

In another aspect, the invention provides a method of identifying non-target-site herbicide resistance in wild grass, the method comprising:
i) determining the level of at least one biomarker selected from the group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1 in a test sample of the wild grass; and
ii) comparing the level of the at least one biomarker in the test sample with the level of the at least one biomarker in a control sample or with a predetermined reference level for the at least one biomarker;
wherein an increased level of the at least one biomarker in the test sample compared to the control sample or compared to the predetermined reference level is indicative of non-target-site herbicide resistance based upon metabolic based resistance.

Optionally, the biomarker is protein or mRNA.

Optionally, the wild grass is selected from the group consisting of black grass, rye grass, wild oat and bent grass.

Optionally, the test sample is a stem sample or a leaf sample.

Optionally, the test sample is obtained post emergence.

Optionally, the control sample is obtained from a herbicide sensitive wild grass of the same species, or the predetermined reference level is the average level of the at least one biomarker in a herbicide sensitive wild grass of the same species.

Optionally, the level of the at least one biomarker in the test sample is increased by at least 1.5 fold, at least 2 fold, at least 2.5 fold, or at least 5 fold compared to the control sample or predetermined reference level.

In another aspect, the invention provides a kit for identifying non-target-site herbicide resistance (NTSR) in wild grass, wherein the NTSR is based upon metabolic based resistance, the kit comprising: a detectably labelled agent that specifically binds to a biomarker selected from the group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1.

Optionally, the biomarker is protein or mRNA.

Optionally, the kit further comprises one or more reagents for detecting the detectably labelled agent.

In another aspect, the invention provides an assay device for identifying non-target-site herbicide resistance (NTSR) of wild grass, wherein the NTSR is based upon metabolic based resistance, the device comprising a surface with a detectably labelled agent located thereon, wherein the detectably labelled agent specifically binds to a biomarker selected from the group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1.

Optionally, the assay device comprises at least two detectably labelled agents located on the surface, wherein the detectably labelled agents specifically bind to different biomarkers selected from the group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1.

Optionally, the at least two detectably labeled agents are located in separate zones on the surface.

Optionally, the biomarker is protein or mRNA.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### BRIEF DESCRIPTION OF THE DRAWINGS

Embodiments of the invention are further described hereinafter with reference to the accompanying drawings, in which:

FIG. 1 shows representative proteome maps for leaf and shoot tissue of Non-Target Site Resistant (NTSR—Peldon, Oxford) and Herbicide Susceptible (HS, Rothamsted) black-grass populations. FIG. 1A shows that protein spots that are significantly over produced (+1.5 fold) in the NTSR populations compared to the HS are numbered and indicated on the maps; FIG. 1B shows two representative protein spots illustrating their increased synthesis in two NTSR populations.

FIG. 2 shows similarities in protein spots differentially synthesised in NTSR populations compared to the HS control, including spots that were induced under other biotic/abiotic stress conditions. The numbers in brackets are the proteins that were not induced under biotic/abiotic stress. The seven proteins that were similarly over synthesised in the Peldon, Oxford, and Pendimethalin populations and not induced under biotic/abiotic stress in HS plants are claimed as novel NTSR biomarkers.

FIG. 3 provides details on the seven new biomarkers that were identified with increased abundance in each of the Peldon, Oxford and Pendimethalin NTSR populations, but not in the Fenoxaprop NTSR population.

FIG. 4 shows the amino acid sequence for Stem-specific protein TSJT1 (SEQ ID NO:1).

FIG. 5 shows the amino acid sequence for 12-oxophytodienoate reductase 1 (SEQ ID NO:2).

FIG. 6 shows the amino acid sequence for D-3-phosphoglycerate dehydrogenase 1 (SEQ ID NO:3).

FIG. 7 shows the amino acid sequence for GSTF2 (SEQ ID NO:4).

FIG. 8 shows the amino acid sequence for GSTU2 (SEQ ID NO:5).

FIG. 9 shows the amino acid sequence for NAD-dependent epimerase/dehydratase (SEQ ID NO:6).

FIG. 10 shows the amino acid sequence for NADPH:quinone oxidoreductase 1 (SEQ ID NO:7).

FIG. 11 shows the nucleotide coding sequence for Stem-specific protein TSJT1 (SEQ ID NO:8).

FIG. 12 shows the nucleotide coding sequence for 12-oxophytodienoate reductase 1 (SEQ ID NO:9).

FIG. 13 shows the nucleotide coding sequence for D-3-phosphoglycerate dehydrogenase 1 (SEQ ID NO:10).

FIG. 14 shows the nucleotide coding sequence for GSTF2 (SEQ ID NO:11).

FIG. 15 shows the nucleotide coding sequence for GSTU2 (SEQ ID NO:12).

FIG. 16 shows the nucleotide coding sequence for NAD-dependent epimerase/dehydratase (SEQ ID NO:13).

FIG. 17 shows the nucleotide coding sequence for NADPH:quinone oxidoreductase 1 (SEQ ID NO:14).

FIG. 18 shows the herbicide resistance profiles of the Fenoxaprop, and Pendimethalin NTSR blackgrass populations and the HS (Roth 09) population, studied herein.

FIG. 19 shows a summary of the whole transcriptomics data.

FIG. 20 shows a comparison of protein levels in Black grass populations with different susceptibility to herbicide (R=Rothamsted=susceptible; P=Peldon+Resistant) by Western blotting. The amount of protein for the four proteins Am12-oxophytodienoate-reductase, AmGstF2, AmGstu2 and AmD-3-phosphoglycerate dehydrogenase was compared in Alopecurus myosuroides Rothamsted (R) and Peldon (P) populations. Fifty micrograms of protein extract from stem was loaded in each lane and analyzed by SDS-PAGE follow by Western blotting using specific antibodies raised against four selected proteins. The arrows indicate the detected band that corresponds to the expected apparent relative molecular mass (Mr) deduced from each protein sequence. The Mr (k) and positions of the markers are indicated.

### DETAILED DESCRIPTION

Transcriptomic and proteomic analysis of four different NTSR black-grass populations was carried out and compared to the transcriptome and proteome of a herbicide susceptible (HS) black grass population. Both field collected populations (“oxford” and “peldon”) and populations that had been experimentally selected for herbicide resistance (“fenoxaprop” and “pendimethalin”) were analysed. Several transcripts and corresponding proteins (spanning metabolic pathways) had increased abundance in NTSR black-grass populations compared to the HS black-grass population.

Further experiments were performed to compare the NTSR black grass proteome with the proteome of HS blackgrass exposed to a range of biotic (insect feeding, plant-microbe interaction) and abiotic stresses (N-limitation, osmotic, heat), including herbicide safening. Surprisingly, it was found that the vast majority of changes in protein abundance associated with NTSR were distinct from the changes in protein abundance observed in response to biotic/abiotic stress. Proteins with a change in abundance in NTSR samples but not HS stressed plants therefore serve as bona fide biomarkers of NTSR.

The data was further analysed to identify proteins with at least a 1.5 fold increased abundance in at least one of the tested NTSR populations compared to HS. As expected, AmGSTF1 was increased in each of the NTSR populations. This is in line with previous findings, where AmGSTF1 was identified as a general biomarker for non-target-site resistance [Cummins et al., 2013].

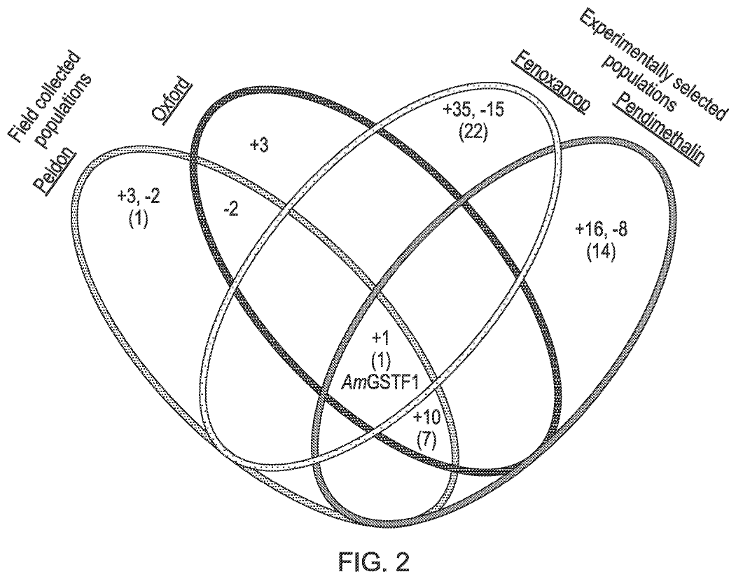

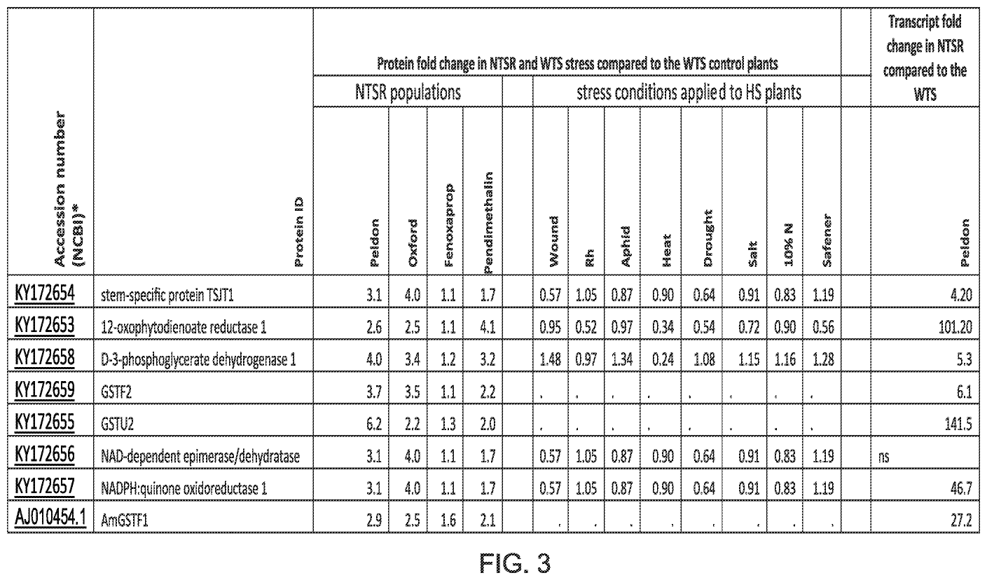

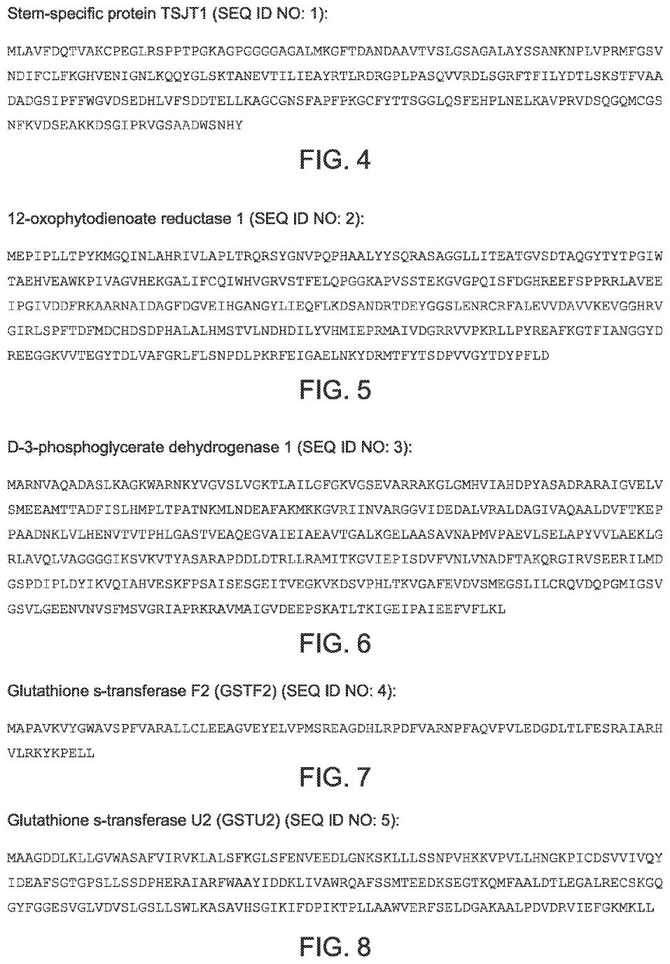

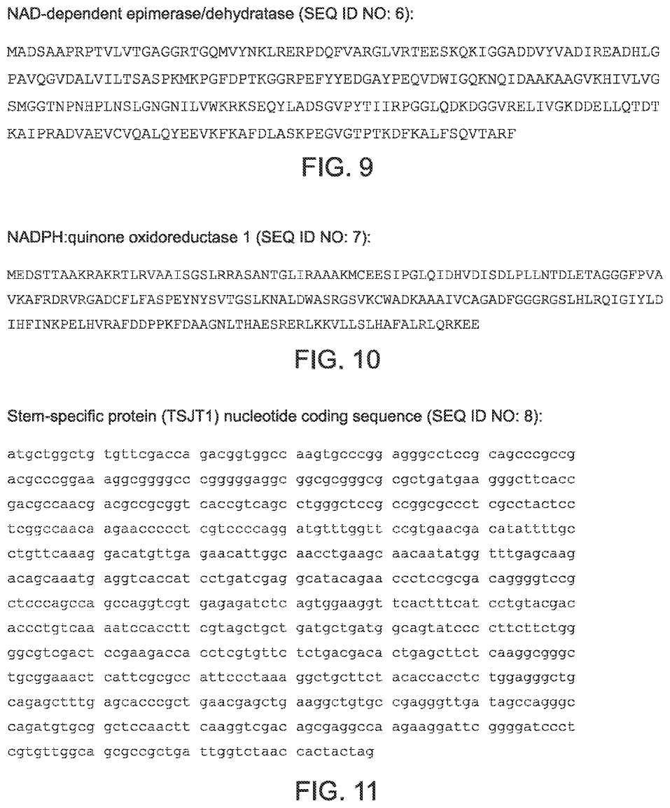

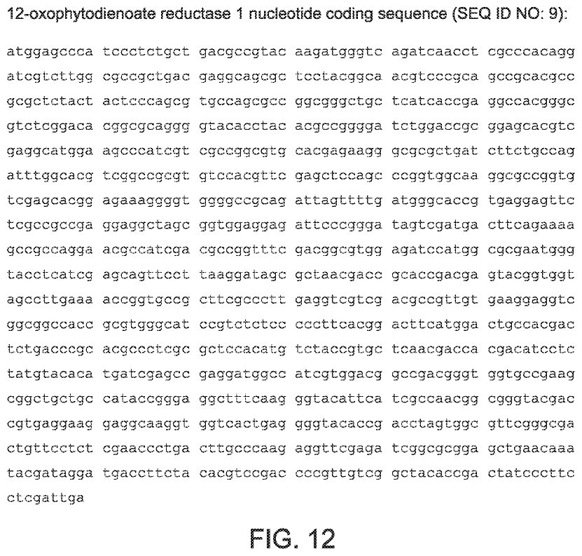

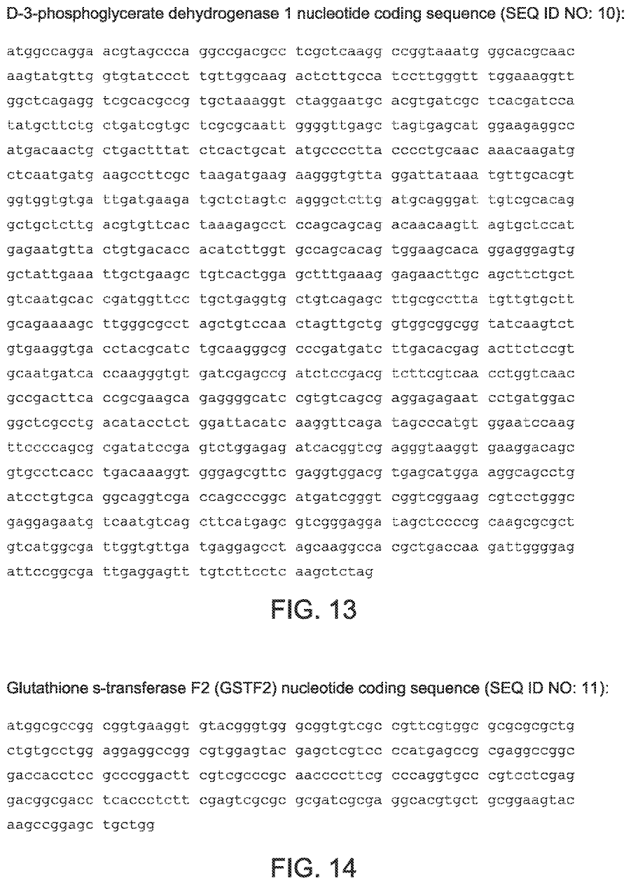

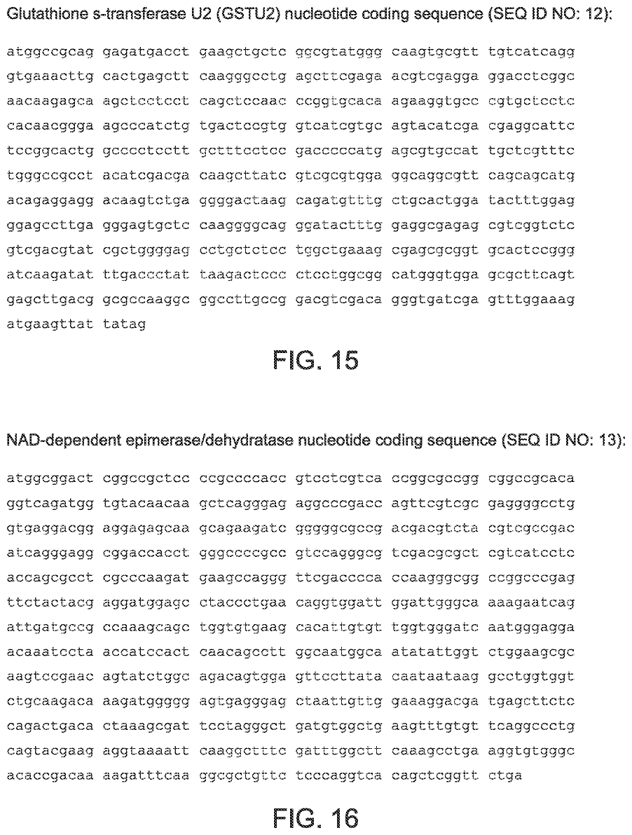

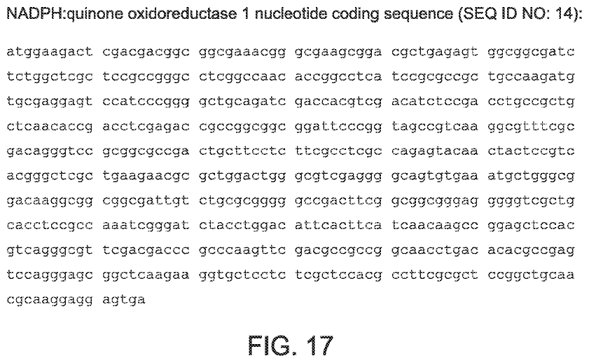

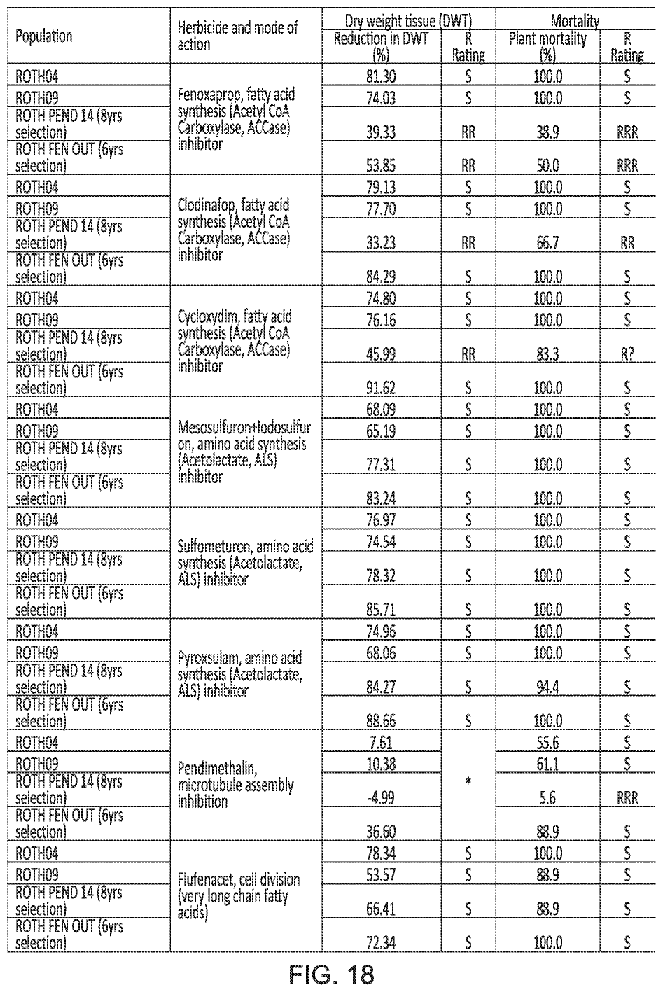

Surprisingly, seven new biomarkers were identified with increased abundance in each of the peldon, oxford and pendimethalin NTSR populations, but not in the fenoxaprop NTSR population (see FIG. 3). Although all of the populations tested display NTSR, the inventors have found that the NTSR in the peldon, oxford and pendimethalin populations is due to metabolic based resistance (and that these populations are resistant against multiple herbicides), whereas the fenoxaprop resistant population appears to arise from the suppression of a fenoxaprop specific toxicity mechanism (and is not resistant against other herbicides) (see FIG. 18). The seven novel biomarkers therefore provide additional valuable information on the type of NTSR present in the tested plant populations. Advantageously, this information can be used to determine the best course of action e.g. whether to change the herbicide used to control the (unwanted) wild grass population, or burn off the crop and wild grass to prevent spread of NTSR wild grass to neighbouring fields.

Use as Biomarkers

Accordingly, in one aspect, the invention relates to the use of at least one of:
i) GSTU2;
ii) D-3-phosphoglycerate dehydrogenase 1;
iii) 12-oxophytodienoate reductase 1;
iv) GSTF2;
NADPH:quinone oxidoreductase 1;
vi) NAD-dependent epimerase/dehydratase;
vii) stem-specific protein TSJT1;

as a biomarker for non-target-site herbicide resistance (NTSR) in wild grass, wherein the NTSR is based upon metabolic based resistance.

The inventors have found that GSTU2, D-3-phosphoglycerate dehydrogenase 1, 12-oxophytodienoate reductase 1, GSTF2, NADPH:quinone oxidoreductase 1, NAD-dependent epimerase/dehydratase, and stem-specific protein TSJT1 are upregulated in the transcriptome and proteome of black grass with NTSR, wherein the NTSR is based upon metabolic based resistance.

Although the invention has been exemplified using black grass, it applies equally to other wild grasses due to the high level of genetic and functional conservation between different wild grass species. Any one of the biomarkers identified in black grass can therefore be used as a biomarker for NTSR based on metabolic based resistance.

As used herein, “biomarker” refers to a naturally occurring molecule, gene or characteristic by which a particular physiological process can be identified. In the context of the invention, a biomarker may be a nucleic acid molecule (typically an mRNA), and/or a protein.

The terms “GSTU2”, “D-3-phosphoglycerate dehydrogenase 1”, “12-oxophytodienoate reductase 1”, “GSTF2”, “NADPH:quinone oxidoreductase 1”, “NAD-dependent epimerase/dehydratase”, and “stem-specific protein TSJT1” therefore refer to the appropriate protein and its corresponding transcript (mRNA). The mRNA can be used as a biomarker. Equally, the corresponding protein can also be used as a biomarker. The terms “peptide”, “protein” and “polypeptide” are used interchangeably herein.

“Stem-specific protein TSJT1” is involved in the regulation of RNA transcription, and may serve to regulate the production of other NTSR protein biomarkers. An example of the amino acid sequence of stem-specific protein TSJT1 in black grass is shown in SEQ ID NO:1. An example of the mRNA sequence of stem-specific protein TSJT1 in black grass is the mRNA sequence that corresponds to the nucleotide coding sequence shown in SEQ ID NO:8. The equivalent protein (and corresponding mRNA transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “stem-specific protein TSJT1” therefore encompasses the specific black grass protein of SEQ ID NO:1 (and the mRNA corresponding to the nucleotide coding sequence of SEQ ID NO:8) as well as functional variants thereof. Such variants may be naturally occurring (e.g. allelic) functional variants of SEQ ID NO:1 or SEQ ID NO:8. The term “variant” also encompasses homologues.

Functional variants will typically contain only conservative substitutions of one or more amino acids of SEQ ID NO:1, or substitution, deletion or insertion of non-critical amino acids in non-critical regions of the protein. A functional variant of SEQ ID NO:1 may therefore be a conservative amino acid sequence variant of SEQ ID NO:1, wherein the variant has stem-specific protein TSJT1 activity.

Equally, functional variants of the mRNA sequence corresponding to the nucleotide coding sequence shown in SEQ ID NO:8 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:8 may therefore be a mRNA variant that encodes a conservative amino acid sequence variant of the protein encoded by SEQ ID NO:8, wherein the encoded conservative amino acid variant has stem-specific protein TSJT1 activity.

Non-functional variants are amino acid sequence variants of SEQ ID NO: 1 that do not have stem-specific protein TSJT1 activity. Non-functional variants will typically contain a non-conservative substitution, a deletion, or insertion or premature truncation of the amino acid sequence of SEQ ID NO:1 or a substitution, insertion or deletion in critical amino acids or critical regions.

Equally, non-functional variants may be mRNA sequence variants of the nucleotide coding sequence shown in SEQ ID NO:8 that do not encode a protein having stem-specific protein TSJT1 activity. Non-functional variants will typically contain a non-conservative nucleotide substitution, a deletion, or insertion or premature truncation of the mRNA sequence corresponding to the nucleotide coding sequence of SEQ ID NO:8 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:1 (or the corresponding functional variants of SEQ ID NO:8). Homologues of stem-specific protein TSJT1 can also be readily identified using standard sequence alignment programmes by a person of ordinary skill in the art. A functional variant of stem-specific protein TSJT1 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:1, or portions or fragments thereof. Suitably, percent identity can be calculated as the percentage of identity to the entire length of the reference sequence (e.g. SEQ ID NO:1), or portions or fragments thereof.

A functional variant of stem-specific protein TSJT1 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:8, or portions or fragments thereof. Suitably, percent identity can be calculated as the percentage of identity to the entire length of the reference sequence (e.g. SEQ ID NO:8), or portions or fragments thereof. “12-oxophytodienoate reductase 1” refers to an enzyme involved in the biosynthesis of the plant hormone jasmonic acid (Sobajima et al., 2003). An example of the amino acid sequence of 12-oxophytodienoate reductase 1 in black grass is shown in SEQ ID NO:2. An example of the mRNA sequence of 12-oxophytodienoate reductase 1 in black grass is the mRNA sequence that corresponds to the nucleotide coding sequence shown in SEQ ID NO:9. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “12-oxophytodienoate reductase 1” therefore encompasses the specific black grass protein (and corresponding transcript) of SEQ ID NO:2 (and the mRNA corresponding to the nucleotide coding sequence of SEQ ID NO:9) as well as functional variants thereof. Such variants may be naturally occurring (e.g. allelic) functional variants of SEQ ID NO:2 or SEQ ID NO:9. The term “variant” also encompasses homologues.

Functional variants will typically contain only conservative substitutions of one or more amino acids of SEQ ID NO:2, or substitution, deletion or insertion of non-critical amino acids in non-critical regions of the protein. A functional variant of SEQ ID NO:2 may therefore be a conservative amino acid sequence variant of SEQ ID NO:2, wherein the variant has 12-oxophytodienoate reductase 1 activity i.e. catalyses the reduction of the cyclic fatty acid derivative 12-oxophytodienoate (OPDA) to 9S,13S-12-oxophytodienoate (9S,13S-OPDA) to 1S,2S-3-oxo-2(2′[Z]-pentenyl)-cyclopentane-1 -octanoate.

Equally, functional variants of the mRNA sequence corresponding to the nucleotide coding sequence shown in SEQ ID NO:9 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:9 may therefore be a mRNA variant that encodes a conservative amino acid sequence variant of the protein encoded by SEQ ID NO:9, wherein the encoded conservative amino acid variant has 12-oxophytodienoate reductase 1 activity.

Non-functional variants are amino acid sequence variants of SEQ ID NO: 2 that do not have 12-oxophytodienoate reductase 1 activity. Non-functional variants will typically contain a non-conservative substitution, a deletion, or insertion or premature truncation of the amino acid sequence of SEQ ID NO:2 or a substitution, insertion or deletion in critical amino acids or critical regions.

Equally, non-functional variants may be mRNA sequence variants of the nucleotide coding sequence shown in SEQ ID NO:9 that do not encode a protein having 12-oxophytodienoate reductase 1 activity. Non-functional variants will typically contain a non-conservative nucleotide substitution, a deletion, or insertion or premature truncation of the mRNA sequence corresponding to the nucleotide coding sequence of SEQ ID NO:9 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:2 (or the corresponding functional variants of SEQ ID NO:89). Homologues of 12-oxophytodienoate reductase 1 can also be readily identified using standard sequence alignment programmes by a person of ordinary skill in the art.

A functional variant of 12-oxophytodienoate reductase 1 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:2, or portions or fragments thereof. Suitably, percent identity can be calculated as the percentage of identity to the entire length of the reference sequence (e.g. SEQ ID NO:2), or portions or fragments thereof.

A functional variant of 12-oxophytodienoate reductase 1 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:9, or portions or fragments thereof. Suitably, percent identity can be calculated as the percentage of identity to the entire length of the reference sequence (e.g. SEQ ID NO:9), or portions or fragments thereof.

“D-3-phosphoglycerate dehydrogenase 1” refers to an enzyme that catalyses the transition of 3-phosphoglycerate into 3-phospohydroxypyruvate, which in the first step in the phosphorylated pathway of L-serine biosynthesis. This is essential in cysteine and glycine synthesis, which lie further downstream in the pathway (Xu et al., 2015). An example of the amino acid sequence of D-3-phosphoglycerate dehydrogenase 1 in black grass is shown in SEQ ID NO:3. An example of the mRNA sequence of D-3-phosphoglycerate dehydrogenase 1 in black grass is the mRNA sequence that corresponds to the nucleotide coding sequence shown in SEQ ID NO:10. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “D-3-phosphoglycerate dehydrogenase 1” therefore encompasses the specific black grass protein (and corresponding transcript) of SEQ ID NO:3 (and the mRNA corresponding to the nucleotide coding sequence of SEQ ID NO:10) as well as functional variants thereof. Such variants may be naturally occurring (e.g. allelic) functional variants of SEQ ID NO:3 or SEQ ID NO:10. The term “variant” also encompasses homologues.

Functional variants will typically contain only conservative substitutions of one or more amino acids of SEQ ID NO:3, or substitution, deletion or insertion of non-critical amino acids in non-critical regions of the protein. A functional variant of SEQ ID NO:3 may therefore be a conservative amino acid sequence variant of SEQ ID NO:3, wherein the variant has D-3-phosphoglycerate dehydrogenase 1 activity.

Equally, functional variants of the mRNA sequence corresponding to the nucleotide coding sequence shown in SEQ ID NO:10 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:10 may therefore be a mRNA variant that encodes a conservative amino acid sequence variant of the protein encoded by SEQ ID NO:10, wherein the encoded conservative amino acid variant has D-3-phosphoglycerate dehydrogenase 1 activity.

Non-functional variants are amino acid sequence variants of SEQ ID NO: 3 that do not have D-3-phosphoglycerate dehydrogenase 1 activity. Non-functional variants will typically contain a non-conservative substitution, a deletion, or insertion or premature truncation of the amino acid sequence of SEQ ID NO:3 or a substitution, insertion or deletion in critical amino acids or critical regions.

Equally, non-functional variants may be mRNA sequence variants of the nucleotide coding sequence shown in SEQ ID NO:10 that do not encode a protein having D-3-phosphoglycerate dehydrogenase 1 activity. Non-functional variants will typically contain a non-conservative nucleotide substitution, a deletion, or insertion or premature truncation of the mRNA sequence corresponding to the nucleotide coding sequence of SEQ ID NO:10 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:3 (or the corresponding functional variants of SEQ ID NO:10). Homologues of D-3-phosphoglycerate dehydrogenase 1 can also be readily identified using standard sequence alignment programmes by a person of ordinary skill in the art.

A functional variant of D-3-phosphoglycerate dehydrogenase 1 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:3, or portions or fragments thereof. Suitably, percent identity can be calculated as the percentage of identity to the entire length of the reference sequence (e.g. SEQ ID NO:3), or portions or fragments thereof.

A functional variant of D-3-phosphoglycerate dehydrogenase 1 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:10, or portions or fragments thereof. Suitably, percent identity can be calculated as the percentage of identity to the entire length of the reference sequence (e.g. SEQ ID NO:10), or portions or fragments thereof.

“GSTF2” refers to the second reported enzyme belonging to the Phi class of glutathione-S-transferases (GST) in black-grass. GSTs catalyse the conjugation of reduced glutathione to a number of exogenous and endogenous electrophiles, for their transport into plant vacuoles, where they are innocuous. An example of the amino acid sequence of GSTF2 in black grass is shown in SEQ ID NO:4. An example of the mRNA sequence of GSTF2 in black grass is the mRNA sequence that corresponds to the nucleotide coding sequence shown in SEQ ID NO:11. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “GSTF2” therefore encompasses the specific black grass protein (and corresponding transcript) of SEQ ID NO:4 (and the mRNA corresponding to the nucleotide coding sequence of SEQ ID NO:11) as well as functional variants thereof. Such variants may be naturally occurring (e.g. allelic) functional variants of SEQ ID NO:4 or SEQ ID NO:11. The term “variant” also encompasses homologues.

Functional variants will typically contain only conservative substitutions of one or more amino acids of SEQ ID NO:4, or substitution, deletion or insertion of non-critical amino acids in non-critical regions of the protein. A functional variant of SEQ ID NO:4 may therefore be a conservative amino acid sequence variant of SEQ ID NO:4, wherein the variant has GSTF2 activity.

Equally, functional variants of the mRNA sequence corresponding to the nucleotide coding sequence shown in SEQ ID NO:11 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:11 may therefore be a mRNA variant that encodes a conservative amino acid sequence variant of the protein encoded by SEQ ID NO:11, wherein the encoded conservative amino acid variant has GSTF2 activity.

Non-functional variants are amino acid sequence variants of SEQ ID NO: 4 that do not have GSTF2 activity. Non-functional variants will typically contain a non-conservative substitution, a deletion, or insertion or premature truncation of the amino acid sequence of SEQ ID NO:4 or a substitution, insertion or deletion in critical amino acids or critical regions.

Equally, non-functional variants may be mRNA sequence variants of the nucleotide coding sequence shown in SEQ ID NO:11 that do not encode a protein having GSTF2 activity. Non-functional variants will typically contain a non-conservative nucleotide substitution, a deletion, or insertion or premature truncation of the mRNA sequence corresponding to the nucleotide coding sequence of SEQ ID NO:11 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:4 (or the corresponding functional variants of SEQ ID NO:11). Homologues of GSTF2 can also be readily identified using standard sequence alignment programmes by a person of ordinary skill in the art.

A functional variant of GSTF2 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:4, or portions or fragments thereof. Suitably, percent identity can be calculated as the percentage of identity to the entire length of the reference sequence (e.g. SEQ ID NO:4), or portions or fragments thereof.

A functional variant of GSTF2 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:11, or portions or fragments thereof. Suitably, percent identity can be calculated as the percentage of identity to the entire length of the reference sequence (e.g. SEQ ID NO:11), or portions or fragments thereof.

“GSTU2” refers to the second reported enzyme belonging to the tau class of glutathione-S-transferases (GST) in black-grass. GSTs catalyse the conjugation of reduced glutathione to a number of exogenous and endogenous electrophiles, for their transport into plant vacuoles, where they are innocuous. An example of the amino acid sequence of GSTU2 in black grass is shown in SEQ ID NO:5. An example of the mRNA sequence of GSTU2 in black grass is the mRNA sequence that corresponds to the nucleotide coding sequence shown in SEQ ID NO:12. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “GSTU2” therefore encompasses the specific black grass protein (and corresponding transcript) of SEQ ID NO:5 (and the mRNA corresponding to the nucleotide coding sequence of SEQ ID NO:12) as well as functional variants thereof. Such variants may be naturally occurring (e.g. allelic) functional variants of SEQ ID NO:5 or SEQ ID NO:12. The term “variant” also encompasses homologues.

Functional variants will typically contain only conservative substitutions of one or more amino acids of SEQ ID NO:5, or substitution, deletion or insertion of non-critical amino acids in non-critical regions of the protein. A functional variant of SEQ ID NO:5 may therefore be a conservative amino acid sequence variant of SEQ ID NO:5, wherein the variant has GSTU2 activity.

Equally, functional variants of the mRNA sequence corresponding to the nucleotide coding sequence shown in SEQ ID NO:12 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:12 may therefore be a mRNA variant that encodes a conservative amino acid sequence variant of the protein encoded by SEQ ID NO:12, wherein the encoded conservative amino acid variant has GSTU2 activity.

Non-functional variants are amino acid sequence variants of SEQ ID NO: 5 that do not have GSTU2 activity. Non-functional variants will typically contain a non-conservative substitution, a deletion, or insertion or premature truncation of the amino acid sequence of SEQ ID NO:5 or a substitution, insertion or deletion in critical amino acids or critical regions.

Equally, non-functional variants may be mRNA sequence variants of the nucleotide coding sequence shown in SEQ ID NO:12 that do not encode a protein having GSTU2 activity. Non-functional variants will typically contain a non-conservative nucleotide substitution, a deletion, or insertion or premature truncation of the mRNA sequence corresponding to the nucleotide coding sequence of SEQ ID NO:12 that results in a substitution, insertion or deletion in critical amino acids or critical regions of the encoded protein. Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:5 (or the corresponding functional variants of SEQ ID NO:12). Homologues of GSTU2 can also be readily identified using standard sequence alignment programmes by a person of ordinary skill in the art.

A functional variant of GSTU2 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:5, or portions or fragments thereof. Suitably, percent identity can be calculated as the percentage of identity to the entire length of the reference sequence (e.g. SEQ ID NO:5), or portions or fragments thereof.

A functional variant of GSTU2 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:12, or portions or fragments thereof. Suitably, percent identity can be calculated as the percentage of identity to the entire length of the reference sequence (e.g. SEQ ID NO:12), or portions or fragments thereof.

“NAD-dependent epimerase/dehydratase” refers to an enzyme domain that domain is found in proteins that utilise NAD as a cofactor and use nucleotide-sugar substrates for a variety of chemical reactions. One of the best studied of these proteins is UDP-galactose 4-epimerase which catalyses the conversion of UDP-galactose to UDP-glucose during galactose metabolism. An example of the amino acid sequence of NAD-dependent epimerase/dehydratase in black grass is shown in SEQ ID NO:6. An example of the mRNA sequence of NAD-dependent epimerase/dehydratase in black grass is the mRNA sequence that corresponds to the nucleotide coding sequence shown in SEQ ID NO:13. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “NAD-dependent epimerase/dehydratase” therefore encompasses the specific black grass protein (and corresponding transcript) of SEQ ID NO:6 (and the mRNA corresponding to the nucleotide coding sequence of SEQ ID NO:13) as well as functional variants thereof. Such variants may be naturally occurring (e.g. allelic) functional variants of SEQ ID NO:6 or SEQ ID NO:13. The term “variant” also encompasses homologues.

Functional variants will typically contain only conservative substitutions of one or more amino acids of SEQ ID NO:6, or substitution, deletion or insertion of non-critical amino acids in non-critical regions of the protein. A functional variant of SEQ ID NO:6 may therefore be a conservative amino acid sequence variant of SEQ ID NO:6, wherein the variant has NAD-dependent epimerase/dehydratase activity.

Equally, functional variants of the mRNA sequence corresponding to the nucleotide coding sequence shown in SEQ ID NO:13 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:13 may therefore be a mRNA variant that encodes a conservative amino acid sequence variant of the protein encoded by SEQ ID NO:13, wherein the encoded conservative amino acid variant has NAD-dependent epimerase/dehydratase activity.

Non-functional variants are amino acid sequence variants of SEQ ID NO: 6 that do not have NAD-dependent epimerase/dehydratase activity. Non-functional variants will typically contain a non-conservative substitution, a deletion, or insertion or premature truncation of the amino acid sequence of SEQ ID NO:6 or a substitution, insertion or deletion in critical amino acids or critical regions.

Equally, non-functional variants may be mRNA sequence variants of the nucleotide coding sequence shown in SEQ ID NO:13 that do not encode a protein having NAD-dependent epimerase/dehydratase activity. Non-functional variants will typically contain a non-conservative nucleotide substitution, a deletion, or insertion or premature truncation of the mRNA sequence corresponding to the nucleotide coding sequence of SEQ ID NO:13 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:6 (or the corresponding functional variants of SEQ ID NO:13). Homologues of NAD-dependent epimerase/dehydratase can also be readily identified using standard sequence alignment programmes by a person of ordinary skill in the art.

A functional variant of NAD-dependent epimerase/dehydratase polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:6, or portions or fragments thereof. Suitably, percent identity can be calculated as the percentage of identity to the entire length of the reference sequence (e.g. SEQ ID NO:6), or portions or fragments thereof.

A functional variant of NAD-dependent epimerase/dehydratase mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:13, or portions or fragments thereof. Suitably, percent identity can be calculated as the percentage of identity to the entire length of the reference sequence (e.g. SEQ ID NO:13), or portions or fragments thereof.

“NADPH:quinone oxidoreductase 1” refers to an enzyme that reduces quinones and xenobiotics to less reactive compounds via 2-electron reduction (Gray et al., 2016). An example of the amino acid sequence of NAD-dependent epimerase/dehydratase in black grass is shown in SEQ ID NO:7. An example of the mRNA sequence of NADPH:quinone oxidoreductase 1 in black grass is the mRNA sequence that corresponds to the nucleotide coding sequence shown in SEQ ID NO:14. However, the equivalent protein (and corresponding transcript) present in other wild grasses may also be used as a biomarker in accordance with the invention. The identification of equivalent proteins (e.g. functional variants) and corresponding transcripts is well within the routine capabilities of a person of ordinary skill in the art. The phrase “NADPH:quinone oxidoreductase 1” therefore encompasses the specific black grass protein (and corresponding transcript) of SEQ ID NO:7 (and the mRNA corresponding to the nucleotide coding sequence of SEQ ID NO:14) as well as functional variants thereof. Such variants may be naturally occurring (e.g. allelic) functional variants of SEQ ID NO:7 or SEQ ID NO:14. The term “variant” also encompasses homologues.

Functional variants will typically contain only conservative substitutions of one or more amino acids of SEQ ID NO:7, or substitution, deletion or insertion of non-critical amino acids in non-critical regions of the protein. A functional variant of SEQ ID NO:7 may therefore be a conservative amino acid sequence variant of SEQ ID NO:7, wherein the variant has NADPH:quinone oxidoreductase 1 activity.

Equally, functional variants of the mRNA sequence corresponding to the nucleotide coding sequence shown in SEQ ID NO:14 will typically contain only substitutions of one or more nucleic acids wherein the substitutions either do not change the encoded amino acid sequence, or result in a conservative amino acid substitution of one or more of the encoded amino acids, or a substitution, deletion or insertion in non-critical regions of the encoded protein. A functional mRNA variant of the nucleotide coding sequence shown in SEQ ID NO:14 may therefore be a mRNA variant that encodes a conservative amino acid sequence variant of the protein encoded by SEQ ID NO14, wherein the encoded conservative amino acid variant has NADPH:quinone oxidoreductase 1 activity.

Non-functional variants are amino acid sequence variants of SEQ ID NO: 7 that do not have NADPH:quinone oxidoreductase 1 activity. Non-functional variants will typically contain a non-conservative substitution, a deletion, or insertion or premature truncation of the amino acid sequence of SEQ ID NO:7 or a substitution, insertion or deletion in critical amino acids or critical regions.

Equally, non-functional variants may be mRNA sequence variants of the nucleotide coding sequence shown in SEQ ID NO:14 that do not encode a protein having NADPH:quinone oxidoreductase 1 activity. Non-functional variants will typically contain a non-conservative nucleotide substitution, a deletion, or insertion or premature truncation of the mRNA sequence corresponding to the nucleotide coding sequence of SEQ ID NO:14 that results in a substitution, insertion or deletion in critical amino acids or critical regions of the encoded protein.

Methods for identifying functional and non-functional variants (e.g. functional and non-functional allelic variants) are well known to a person of ordinary skill in the art. Accordingly, a person of skill in the art would readily be able to identify amino acids that may be substituted to provide functional variants (or functional fragments), such as conservative amino acid sequence variants, of SEQ ID NO:7 (or the corresponding functional variants of SEQ ID NO:14). Homologues of NADPH:quinone oxidoreductase 1 can also be readily identified using standard sequence alignment programmes by a person of ordinary skill in the art.

A functional variant of NADPH:quinone oxidoreductase 1 polypeptide may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:7, or portions or fragments thereof. Suitably, percent identity can be calculated as the percentage of identity to the entire length of the reference sequence (e.g. SEQ ID NO:7), or portions or fragments thereof.

A functional variant of NADPH:quinone oxidoreductase 1 mRNA may comprise a nucleotide sequence having at least about 60%, 65%, 70%, 75%, 80%, 85%, 90%, 91%, 92%, 93%, 94%, 95%, 96%, 97%, 98%, 99% or 100% identity to the mRNA sequence corresponding to the nucleotide coding sequence of SEQ ID NO:14, or portions or fragments thereof. Suitably, percent identity can be calculated as the percentage of identity to the entire length of the reference sequence (e.g. SEQ ID NO:14), or portions or fragments thereof.

A “non-essential” or “non-critical” amino acid residue is a residue that can be altered from the wild-type sequence (e.g., the sequence of SEQ ID NO:1 to 7) without abolishing or, more preferably, without substantially altering biological activity, whereas an “essential” amino acid residue results in such a change. For example, amino acid residues that are conserved among the polypeptides of the present invention are predicted to be particularly non-amenable to alteration, except that amino acid residues in transmembrane domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in protein is preferably replaced with another amino acid residue from the same side chain family. Alternatively, in another embodiment, mutations can be introduced randomly along all or part of coding sequences, such as by saturation mutagenesis, and the resultant mutants can be screened for biological activity to identify mutants that retain activity. Following mutagenesis of SEQ ID NO:1 to 7, the encoded proteins can be expressed recombinantly and the biological activity of the protein can be determined.

As used herein, a “biologically active portion” of protein or a protein portion with “biological activity” includes fragment of protein that participate in an interaction between molecules and non-molecules. Biologically active portions of protein include peptides comprising amino acid sequences sufficiently homologous to or derived from the amino acid sequences of the protein, e.g., the amino acid sequences shown in SEQ ID NO: 1 to 7, which include fewer amino acids than the full length protein, and exhibit at least one activity of the encoded protein. Typically, biologically active portions comprise a domain or motif with at least one activity of the protein.

A biologically active portion of protein can be a polypeptide that is, for example, 50, 100, 150, 200, 250, 300, 350, 400, 450, 500 or more amino acids in length of SEQ ID NO:1 to 7.

Biologically active portions of protein can be used as targets for developing agents that modulate mediated activities, e.g., biological activities described herein.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides described herein can have amino acid sequences sufficiently or substantially identical to the amino acid sequences of SEQ ID NO:1 to 7. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

The invention is directed to the use of at least one (i.e. one or more) of the above mentioned biomarkers for the detection of NTSR based upon metabolic based resistance. The terms “at least one” and “one or more” are used to encompass one, two, three, four, five, six or seven of the mentioned biomarkers (at either the mRNA or protein level or a mixture thereof) in any combination. The invention therefore encompasses a panel of two or more, three or more, four or more, five or more, six or more, or seven of the biomarkers (at either the mRNA or protein level or a mixture thereof) in any combination.

In the context of the invention, the one or more biomarkers are useful for determining NTSR in wild grass. The term “grass” is well known in the art and refers to any monocotyledonous plant of the family Poaceae (formerly Gramineae), having jointed stems and sheathed by long narrow leaves, flowers in spikes and seedlike fruits. The family is chiefly herbaceous but includes some woody plants. Examples of grasses include but are not limited to cereals, bamboo, reeds, sugar cane.

The term “wild grass” refers to a “grass” as defined above, wherein the grass has not been intentionally planted or grown (and typically grows unwanted) in a controlled setting such as a field e.g. a crop field.

In one embodiment, a wild grass is selected from the group consisting of black grass, rye grass, wild oat and bent grass. Other wild grasses to which the invention may apply include A) members of the genus Bromus notably sterile brome (B. sterilis), great brome (B. diandrus), soft brome (B. hordeaceus), mesdow brome (B. commutatus) and rye brome (B. secalinus); B) members of the genus Echinochloa such as jungle rice (Echinochloa colona), and barnyardgrass (Echinochloa crus-galli); C) members of the genus Zizania wild rice species.

“Black grass” is the common name for Alopecurus myosuroides. It is a wild grass that is well known as a damaging weed in crop fields such as wheat. The invention also extends to all other species of Alopecurus, notably

1. Alopecurus aequalis

2. Alopecurus albovii

3. Alopecurus anatolicus

4. Alopecurus apiatus

5. Alopecurus arundinaceus

6. Alopecurus aucheri

7. Alopecurus baptarrhenius

8. Alopecurus bonariensis

9. Alopecurus borii

10. Alopecurus bornmuelleri

11. Alopecurus brachystachus

12. Alopecurus bulbosus

13. Alopecurus carolinianus

14. Alopecurus creticus

15. Alopecurus dasyanthus

16. Alopecurus davisii

17. Alopecurus geniculatus

18. Alopecurus gerardii

19. Alopecurus glacialis

20. Alopecurus x haussknechtianus

21. Alopecurus heliochloides

22. Alopecurus himalaicus

23. Alopecurus hitchcockii

24. Alopecurus japonicus

25. Alopecurus laguroides

26. Alopecurus lanatus

27. Alopecurus longiaristatus

28. Alopecurus magellanicus

29. Alopecurus x marssonii

30. Alopecurus mucronatus

31. Alopecurus myosuroides

32. Alopecurus nepalensis

33. Alopecurus x plettkei

34. Alopecurus ponticus

35. Alopecurus pratensis

36. Alopecurus rendlei

37. Alopecurus saccatus

38. Alopecurus setarioides

39. Alopecurus textilis

40. Alopecurus turczaninovii

41. Alopecurus x turicensis

42. Alopecurus utriculatus

43. Alopecurus vaginatus

44. Alopecurus x winklerianus

“Rye grass” or “perennial rye grass” is the common name for Lolium perenne. Also known as English ryegrass or winter ryegrass, is a grass from the family Poaceae. It is native to Europe, Asia and northern Africa. The invention also extends to other Loilium species notably
1. Lolium arundinaceum (Schreb.) Darbysh.
2. Lolium canariense Steud.
3. Lolium giganteum Lam.
4. Lolium x hybridum Hausskn
5. Lolium mazzettianum (E. B. Alexeev) Darbysh.
6. Lolium multiflorum Lam.
7. Lolium perenne L.
8. Lolium persicum Boiss. & Hohen.
9. Lolium pratense (Huds.) Darbysh.
10. Lolium remotum Schrank
11. Lolium rigidum Gaudin
12. Lolium saxatile H. Scholz & S. Scholz
13. Lolium temulentum L.

“Wild oat” or “oat-grasses” are species of the Avena genus that occur in the wild, particularly as weeds in agricultural fields. Examples of wild oat species include, but are not limited to Avena aemulans, Avena barbata, Avena brevis, Avena chinensis, Avena clauda, Avena eriantha, Avena fatua, Avena longiglumis, Avena maroccana, Avena murphyi, Avena prostrata, Avena saxatilis, Avena sterilis Avena strigosa, Avena vaviloviana, Avena ventricosa, and Avena volgensis.

“Bent grass” is the common name for plants from the Agrostis genus, which includes hundreds of species such as, Agrostis aequivalvi, Agrostis agrostiflora, Agrostis alpina, Agrostis ambatoensis, Agrostis anadyrensis, Agrostis angrenica, Agrostis arvensis, Agrostis Atlantica, Agrostis australiensis, Agrostis bacillata, Agrostis balansae, Agrostis barceloi, Agrostis basalis, Agrostis bergiana, Agrostis bettyae, Agrostis xbjoerkmannii, Agrostis blasdalei, Agrostis boliviana, Agrostis boormanii, Agrostis bourgaei, Agrostis boyacensis, Agrostis brachiata, Agrostis brachyathera, Agrostis breviculmis, Agrostis burmanica, Agrostis calderoniae, Agrostis canina, Agrostis capillaris, Agrostis carmichaelii, Agrostis castellana, Agrostis x castriferrei, Agrostis clavata, Agrostis xclavatiformis, Agrostis clemensorum, Agrostis comorensis, Agrostis congestiflora, Agrostis continuata, Agrostis curtisii, Agrostis cypricola, Agrostis decaryana, Agrostis delicatula, Agrostis delislei, Agrostis densiflora, Agrostis diemenica, Agrostis dimorpholemma, Agrostis divaricatissima, Agrostis dshungarica, Agrostis durieui, Agrostis dyeri, Agrostis elliotii, Agrostis elliottiana, Agrostis emirnensis, Agrostis eriantha, Agrostis exarata Agrostis exserta, Agrostis filipes, Agrostis flaccida, Agrostis foliata, Agrostis x fouilladeana, Agrostis gelida, Agrostis ghiesbreghtii, Agrostis gigantea, Agrostis x gigantifera, Agrostis glabra, Agrostis goughensis, Agrostis gracilifolia, Agrostis gracililaxa, Agrostis griffithiana, Agrostis hallii, Agrostis x hegetschweileri, Agrostis hendersonii, Agrostis hesperica, Agrostis hideoi, Agrostis hirta, Agrostis holgateana, Agrostis hookeriana, Agrostis hooveri, Agrostis howellii, Agrostis hugoniana, Agrostis humbertii, Agrostis humilis, Agrostis hyemalis, Agrostis hygrometrica, Agrostis idahoensis, Agrostis imbecilla, Agrostis imberbis, Agrostis inaequiglumis, Agrostis inconspicua, Agrostis infirma, Agrostis innominata, Agrostis insularis, Agrostis isopholis, Agrostis jahnii, Agrostis joyceae, Agrostis juressii, Agrostis keniensis, Agrostis kilimandscharica, Agrostis koelerioides, Agrostis kolymensis, Agrostis korczaginii, Agrostis lacuna-vernalis, Agrostis laxissima, Agrostis lazica, Agrostis lehmannii, Agrostis lenis, Agrostis leptotricha, Agrostis liebmannii, Agrostis longiberbis, Agrostis mackliniae, Agrostis magellanica, Agrostis mannii, Agrostis marojejyensis, Agrostis masafuerana, Agrostis media, Agrostis mertensii, Agrostis merxmuelleri, Agrostis meyenii, Agrostis micrantha, Agrostis microphylla, Agrostis montevidensis, Agrostis muelleriana, Agrostis munroana, Agrostis x murbeckii, Agrostis muscosa, Agrostis musjidii, Agrostis nebulosa, Agrostis nervosa, Agrostis nevadensis, Agrostis nevskii, Agrostis nipponensis, Agrostis novogaliciana, Agrostis x novograblenovii, Agrostis olympica, Agrostis oregonensis, Agrostis oresbia, Agrostis pallens, Agrostis pallescens, Agrostis x paramushirensis, Agrostis parviflora, Agrostis paulsenii, Agrostis peninsularis, Agrostis perennans, Agrostis personata, Agrostis peschkovae, Agrostis petriei, Agrostis philippiana, Agrostis pilgeriana, Agrostis pilosula, Agrostis pittieri, Agrostis platensis, Agrostis pleiophylla, Agrostis pourretii, Agrostis producta, Agrostis propinqua, Agrostis quinqueseta, Agrostis reuteri, Agrostis rosei, Agrostis rossiae, Agrostis rupestris, Agrostis salaziensis, Agrostis salsa, Agrostis sandwicensis, Agrostis x sanionis, Agrostis scabra, Agrostis scabrifolia, Agrostis schaffneri, Agrostis schleicheri, Agrostis schmidii, Agrostis sclerophylla, Agrostis serranoi, Agrostis sesquiflora, Agrostis sichotensis, Agrostis sikkimensis, Agrostis sinocontracta, Agrostis sinorupestris, Agrostis x stebleri, Agrostis stolonifera, Agrostis x subclavata, Agrostis subpatens, Agrostis subrepens, Agrostis subulata, Agrostis subulifolia, Agrostis tandilensis, Agrostis tateyamensis, Agrostis taylorii, Agrostis tenerrima, Agrostis thompsoniae, Agrostis thurberiana, Agrostis tibestica, Agrostis tileni, Agrostis tolucensis, Agrostis x torgesii, Agrostis trachychlaena, Agrostis trachyphylla, Agrostis trichodes, Agrostis trisetoides, Agrostis tsaratananensis, Agrostis tsiafajavonensis, Agrostis tsitondroinensis, Agrostis turrialbae, Agrostis tuvinica, Agrostis uliginosa, Agrostis umbellata, Agrostis ushae, Agrostis x ussuriensis, Agrostis variabilis, Agrostis venezuelana, Agrostis venusta, Agrostis vidalii, Agrostis vinealis, Agrostis virescens, Agrostis volkensii, Agrostis wacei and Agrostis zenkeri.

“Wild rice” (also known as Canada rice, Indian rice and water oats) refers to four species of grasses form the genus Zizania. Three species of wild rice are native to North America (Zizania palustris, Z. aquatica and Z. texana), with one species native to Asia (Z. latifolia).

The one or more biomarkers described herein are used to determine whether a wild grass has (e.g. harbours, has a phenotype of, displays) a particular type of herbicide resistance (i.e. non-target site herbicide resistance that is based upon metabolic based resistance).

As used herein, “herbicide resistance” refers to the acquired (i.e. inherited) ability of a plant (e.g. a wild grass) to survive (e.g. grow and optionally reproduce) following exposure to a dose of herbicide normally lethal to the wild type. In other words, the plant has phenotypically changed compared to the wild type such that it is not controlled by the herbicide. In a plant, herbicide resistance may be naturally occurring or induced by such techniques as genetic engineering or selection of variants produced by tissue culture or mutagenesis. A “herbicide resistant” plant is therefore a plant that is resistant to (or tolerant to) at least one herbicide at a level that would normally kill, or inhibit the growth of, a wild type plant of the same species. As used herein, “herbicide resistant” and “herbicide tolerant” are used interchangeably. This is also the case for the terms “herbicide resistance” and “herbicide tolerance”.

The level of herbicide resistance in a plant can be determined using any one of several known methods in the art. For example, a plant may be considered herbicide resistant if it survives the application of a herbicide that is applied in accordance with the manufacturers recommendations (i.e. a recommended application that is intended to effectively control or kill the target wildtype (herbicide susceptible) plant). Alternatively, or in addition, a plant may be considered herbicide resistant if it can survive a much higher concentration (e.g. 10% higher, 20% higher, 30% higher, 40% higher, 50% higher, 100% higher etc) of the herbicide than that recommended by the manufacturer.

“Herbicide resistance” as defined above can be determined using several standard methods known in the art. The specific method used may depend on the herbicide in question. A summary of appropriate methods for determining resistance can be found in (Panozzo et al 2015. In one embodiment, herbicide resistant plants display a greater than 50% survival rate compared to wild-type susceptible plants of the same species when any of these methods are performed.

Herbicide resistance is typically measured as an increase in the ability of a plant to survive (and optionally reproduce) following exposure to a particular dose of herbicide, wherein the ability to survive (and optionally reproduce) is compared to a wildtype plant subjected to the same conditions. In this context, the terms “wildtype”, “wildtype susceptible”, “VVTS”, “herbicide susceptible”, “HS” and “control” are used interchangeably to refer to a plant of the same species as the plant being tested for resistance, wherein the “wildtype”, “wildtype susceptible”, “VVTS”, “herbicide susceptible”, “HS” or “control” plant is susceptible (e.g. does not survive) following exposure to the herbicide at the recommended dose. By way of example only, an “increase” in the ability of a plant to survive may be measured as a reduced effect of the herbicide on plant growth and reproduction. The level of reduced effect is related to the plants level of resistance.

The one or more biomarkers described herein are used as markers of non-target site herbicide resistance, wherein the NTSR is based upon metabolic based resistance.

As used herein, the term “NTSR” refers to a non-target-site resistance phenotype in the wild grass. As described in the background section above, NTSR is a general term used to refer to all forms of herbicide resistance for which the underlying cause of resistance is not through mutation in a target site of herbicide action. In other words, NTSR refers to herbicide resistance that is due to (i.e. arises from) an alteration in the plant (e.g. an alteration in one or more molecular mechanism(s) of the plant) that does not directly result from mutation of the target site of herbicide action.

NTSR arises through the avoidance of irreversible cellular damage brought about through detoxification, exclusion, sequestration, suppression of down-stream toxicity or a combination of these cytoprotective responses following chemical exposure. NTSR may therefore result from one or more mechanisms, including enhanced herbicide metabolism (i.e. NTSR based upon metabolic based resistance), reduced herbicide penetration through leaf tissue, target enzyme overproduction, protection against oxidative stress (oxidases, peroxidases), and suppression of herbicide toxicity.

The specific mechanism underlying the NTSR phenotype of a plant population may result in a general defence to herbicides (irrespective of the herbicide's specific mode of action). By way of example, NTSR based upon metabolic based resistance typically provides a general defence to herbicides and as such the NTSR population can be resistant to multiple classes of herbicides. Herbicides may be considered “distinct” when they are from different herbicide classes, and/or they have different target sites in the plants, and/or have different modes of action.

Alternatively, the underlying mechanism of the NTSR phenotype may result in a more specific defence to one herbicide (or a class of herbicides with a common mode of action). By way of example, populations with NTSR arising from suppression of a specific mechanism of herbicide toxicity are only resistant to the herbicide with that mode of action, and therefore have a single herbicide resistance phenotype. Alternatively, populations with NTSR arising from suppression of general or multiple mechanisms of herbicide toxicity are resistant to two or more herbicides, which may have multiple modes of action (these plants are considered to have cross herbicide resistance via multiple resistance mechanisms).

NTSR based upon metabolic based resistance refers to NTSR that arises from mechanisms based on the plants increased production of proteins that aid the plant to tolerate herbicide exposure. This includes “enhanced herbicide detoxification” wherein, there is an enhanced production of proteins that detoxify the herbicide, rendering the herbicides deactivated. . Enhanced herbicide detoxification may be represented by at least a 5%, at least a 10%, at least a 15%, at least a 20%, at least a 30%, at least a 40%, at least a 50%, at least a 60%, at least a 70%, at least a 805, at least a 90% etc. increase in the rate at which (or extent to which) the herbicide is detoxified compared to herbicide sensitive plants. A second mechanism of metabolic based resistance is that of “enhanced stress tolerance” wherein, there is an enhanced production of proteins that enable the plant to tolerate the toxic effects of herbicide exposure, for example by overproducing proteins involved in the plants normal growth and metabolism that is targeted by the herbicide.

The terms “herbicide” and “weedkiller” are used interchangeably herein to refer to chemical substances that are used to control plants.

Herbicides can be classified in many different ways, such as by their selectivity, persistence, means of uptake, intended outcome, chemical family or timing of application. “Selective” herbicides control specific plant species while leaving other plants unharmed. “Non-selective”, also known as “total weedkillers”, typically can control all plant types. “Persistence” is also known as the herbicide's residual action, or how long the herbicide can remain in place and can stay active. This can be reduced by, for example rainfall or reactions with the soil. “Means of uptake” refers to the route used to enter the plant, such as through the roots, where the herbicide is usually applied to the soil, or through foliage, where the herbicide is applied above ground to the plant foliage. The “intended outcome” can also be used to classify herbicides (e.g. where the intended outcome is to be used for the control of weeds, to cause damage to unwanted plants, to suppress specific plants or weeds or by an intention to do something specific to a target plant, such as to act as a defoliant). Herbicides can also be classified by the chemical structure of the active ingredient of the herbicide, either by the whole structure of parts, such as functional groups. The “timing of application” can also be used to classify herbicides, examples include preplant, applied to the soil before other plants; pre-emergence, applied before seedlings of the unwanted plant emerge through the soil surface; or postemergence, applied once the unwanted plant has emerged.

Herbicides can also be classified by their mechanism of action (also known as “mode of action”, or “MOA”). Herbicides classified together by the MOA will likely have the same outcome on the same plants. Farmers with an understanding of this can manage crop and weed resistance to herbicides, selecting the most appropriate for the desired outcome. Classification by MOA can indicate the first enzyme, protein, or biochemical step affected in the plant following application. For example, herbicides can be classified as:

ACCase inhibitors—these typically compounds kill grasses. Acetyl coenzyme A carboxylase (ACCase) is part of the first step of lipid synthesis, they are inhibitors of fatty acid synthesis. Thus, ACCase inhibitors affect cell membrane production in the meristems of the grass plant. In general, broadleaf species are naturally resistant to FOPs, DIMs, and DENs herbicides because of a less sensitive ACCase enzyme. However, ACCase inhibiting herbicides may cause symptoms on certain broadleaf crops. Generally, ACCases of grasses are sensitive to these herbicides, whereas the ACCases of dicot plants are not. The Natural tolerance of some grasses is due to a less sensitive ACCase enzyme or a higher rate of metabolic degradation. These herbicides are absorbed through the foliage and translocated in the phloem to the growing point, where they inhibit meristematic activity. ACCase Inhibitors include herbicides belonging to Aryloxyphenoxypropionate (FOPs), cyclohexanedione (DIMs), and phenylpyrazolin (DENs) chemistries. Fenpxaprop, Clodinafop (Clodinafop-propargyl), Cycoxydim are examples of herbicides which act as ACCase inhibitors.

ALS inhibitors—the acetolactate synthase (ALS) enzyme (also known as acetohydroxyacid synthase, or AHAS) is the first step in the synthesis of the branched-chain amino acids (valine, leucine, and isoleucine). ALS inhibiting herbicides have a broad spectrum of selectivity and are used at low rates as soil-applied or postemergence treatments in many cropping systems, trees and vines, roadsides, range and pasture, turf, and vegetation management. ALS herbicides are readily absorbed by both roots and foliage and translocated in both the xylem and phloem to the site of action at the growing points. ALS has diverse herbicides belonging to different chemistries including: imidazolinones, pyrimidinylthiobenzoates, sulfonylaminocarbonyltriazolinones, sulfonylureas, and triazolopyrimidines. These herbicides inhibit acetolactate synthase, a key enzyme in the pathway of biosynthesis of the branched-chain amino acids isoleucine, leucine, and valine. Plant deaths result from events occurring in response to inhibition of branched-chain amino acids, but the actual sequence of phytotoxic processes is unclear. Mesosulfuron-methyl and iodosulfuron-methyl-sodium (‘mesosulfuron+iodosulfuron’) are used as an ALS-inhibitor herbicide. Sulfometuron and Pyroxsulam are also ALS inhibiting herbicides.

Lipid synthesis inhibitors—herbicides in this mode of action are most effective on annual grasses and some broadleaf weeds. In general, these herbicides are applied preplanting or preemergence and incorporated into the soil. Most herbicides in this mode of action are volatile and need to be incorporated immediately after application to avoid excessive vapor loss. These herbicides are absorbed through both roots and emerging shoots but are translocated only in the xylem. The primary site of absorption and action is the emerging shoot and growing point. Herbicides in this mode of action belong to four chemistries including benzofuranes, chlorocarbonic acids, phosphorodithioates, and thiocarbamates. The specific mode of action of these herbicides is not well elucidated, but there is strong evidence that they interfere with biosynthesis of fatty acids and lipids in the newly developing shoot, which may account for reported reductions in cuticular wax deposition. In addition, these herbicides cause abnormal cell development or prevent cell division in germinating seedlings. They stop the plant from growing by inhibiting cell division in the shoot and root tips while permitting other cell duplication processes to continue.

Microtubule inhibitors—these herbicides are mitotic poisons that inhibit cell division, thus are also known as cell division inhibitors. Thus, the meristematic regions, such as the growing points of stems and roots, are most affected. These are generally applied preemergence to control annual grasses and some broadleaf weeds in many crops and turf grass. These herbicides are absorbed by both roots and shoots of emerging seedlings but are not readily translocated. The emerging shoot is the primary absorption and action site in grass species. Benzamide, benzoic acid (DCPA), dinitroaniline, phosphoramidate, and pyridine herbicides are examples of herbicides that bind to tubulin, the major protein needed to polymerize microtubules that are essential for cell division. Herbicide-induced microtubule loss may cause the observed swelling of root tips as cells in this region neither divide nor elongate. Pendimethalin is an example of a herbicide which inhibits microtubule assembly.

Long chain fatty acid inhibitors—preemergent herbicides that are used to control annual grasses and some small-seeded broadleaf weeds in a variety of crops. They do not control or seriously damage emerging plants. The primary site of absorption and action of these herbicides on broadleaf species is the roots, while the primary site of absorption and action on grass species is the emerging shoot. Long Chain Fatty Acid Inhibitors are not readily translocated in the plant. Long Chain Fatty Acid Inhibitors include acetamide, chloroacetamide, oxyacetamide, and tetrazolinone herbicides that are currently thought to inhibit very long chain fatty acid (VLCFA) synthesis. These compounds typically affect susceptible weeds before emergence but do not inhibit seed germination. Flufenacet is an example of a herbicide which inhibits very long chain fatty acids, which is thought to inhibit cell division.

EPSPS inhibitors—the enolpyruvylshikimate 3-phosphate synthase enzyme EPSPS is used in the synthesis of the amino acids tryptophan, phenylalanine and tyrosine. They affect grasses and dicots alike. Glyphosate (Roundup) is a systemic EPSPS inhibitor inactivated by soil contact.

Synthetic auxins—these inaugurated the era of organic herbicides when discovered in the 1940s after a long study of the plant growth regulator auxin. Synthetic auxins mimic this plant hormone. They have several points of action on the cell membrane, and are effective in the control of dicot plants. 2,4-D is a synthetic auxin herbicide.

Photosystem I inhibitors—these steal electrons from the normal pathway through FeS to Fdx to NADP leading to direct discharge of electrons on oxygen. As a result, reactive oxygen species are produced and oxidation reactions in excess of those normally tolerated by the cell occur, leading to plant death. Bipyridinium herbicides (such as diquat and paraquat) inhibit the Fe-S-Fdx step of that chain, while diphenyl ether herbicides (such as nitrofen, nitrofluorfen, and acifluorfen) inhibit the Fdx-NADP step.

Photosystem II inhibitors—these reduce electron flow from water to NADPH2+ at the photochemical step in photosynthesis. They bind to the Qb site on the D1 protein, and prevent quinone from binding to this site. Therefore, this group of compounds causes electrons to accumulate on chlorophyll molecules. As a consequence, oxidation reactions in excess of those normally tolerated by the cell occur, and the plant dies. The triazine herbicides (including atrazine) and urea derivatives (diuron) are photosystem II inhibitors.

HPPD inhibitors—these inhibit 4-Hydroxyphenylpyruvate dioxygenase, which are involved in tyrosine breakdown. Tyrosine breakdown products are used by plants to make carotenoids, which protect chlorophyll in plants from being destroyed by sunlight. If this happens, the plants turn white due to complete loss of chlorophyll, and the plants die. Mesotrione and sulcotrione are herbicides in this class; a drug, nitisinone, was discovered in the course of developing this class of herbicides.

Carotenoid biosynthesis inhibitors—these herbicides applied as preemergence or postemergence in different cropping systems, landscape and ornamental, industrial use, and aquatic setting. In general, they are active on broadleaf weeds but control selected grasses. The Carotenoid Biosynthesis Inhibiting herbicides interfere directly or indirectly with carotenoid production that protects chlorophyll from excessive light and photo oxidation.

Cellulose inhibitors (cell wall synthesis)—herbicides in this group prevent cell division primarily in developing root tips and are only effective on some germinating broadleaves and selected grasses. Alkylazine, benzamides, and nitriles are Cellulose Inhibitor herbicides that affect cell wall biosynthesis (cellulose) in susceptible plants causing inhibition of cell division.

Glutamine synthesis inhibitors—The glutamine synthetase herbicides inhibit activity of glutamine synthetase, the enzyme that converts glutamate and ammonia to glutamine. This inhibition results in massive accumulation of ammonia in a plant which destroys cells and directly inhibits photosystem I and photosystem II reactions. High ammonia in plants reduces the pH gradient across the membranes which inhibits energy production needed to support plant growth and development. Glufosinate (the only commercialized glutamine synthetase herbicide in the United States) belongs to phosphinic acid chemistry. It is a broad-spectrum postemergent herbicide that controls most annual grasses and broadleaves. It is a contact herbicide with limited translocation throughout the plant.

Protoporphyrinogen oxidase (PPO) inhibitors—Protoporphyrinogen oxidase (PPO) is an enzyme in the chloroplast cell that oxidizes protoporphyrinogen IX (PPGIX) to produce protoporphyrin IX (PPIX). PPIX is important because it is a precursor molecule for both chlorophyll (needed for photosynthesis) and heme (needed for electron transfer chains). Inhibitors of the oxidase enzyme, however, do more than merely block the production of chlorophyll and heme. The inhibition of PPO by inhibitors also results in forming highly reactive molecules that attack and destroy lipids and protein membranes. When a lipid membrane is destroyed, cell becomes leaky and cell organelles dry and disintegrate rapidly. PPO Inhibitors have limited translocation in plants and sometimes are referred to as contact herbicides. PPO Inhibitors injure mostly broadleaf plants; however, certain PPO Inhibitors have some activity on grasses. PPO Inhibitors usually burn plant tissues within hours or days of exposure. PPO Inhibitors used in the United States belong to eight different chemistries including diphenylethers, N-phenylphthalimides, oxadiazoles, oxazolidinediones, phenylpyrazoles, pyrimidindiones, thiadiazoles, and triazolinones. These herbicides are used to control weeds in field crops, vegetables, tree fruits and vines, small fruits, nurseries, lawns, and industry.

Methods of Identifying NTSR in Wild Grass

As described above, the invention is based on the surprising finding that GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”) are each useful biomarkers for NTSR in wild grass, wherein the NTSR is based upon metabolic based resistance .

Accordingly, in one aspect, the invention provides a method of identifying non-target-site herbicide resistance in wild grass, the method comprising:
i) determining the level of at least one biomarker selected from the group consisting of: GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”) in a test sample of the wild grass; and
ii) comparing the level of the at least one biomarker in the test sample with the level of the at least one biomarker in a control sample or with a predetermined reference level for the at least one biomarker;

wherein an increased level of the at least one biomarker in the test sample compared to the control sample or compared to the predetermined reference level is indicative of non-target-site herbicide resistance based upon metabolic based resistance.

The presence, level or absence of biomarker polypeptide or nucleic acid molecule (e.g. mRNA) in a sample from wild grass can be determined by obtaining a sample from the wild grass and contacting the sample with a compound or an agent capable of specifically detecting (e.g. specifically binding) the specific biomarker polypeptide or nucleic acid molecule.

As used herein, the terms “sample”, “test sample” and “sample from wild grass” are used interchangeably to refer to plant tissues, cells, and organs isolated from a wild grass plant, and plant tissues, cells and organs present within a wild grass plant. In one embodiment, the sample is a stem sample or a leaf sample. For the avoidance of doubt, a “stem sample” (also referred to as a “shoot sample” herein) refers to a sample that comprises stem tissue—it does not necessarily, but can, consist of stem tissue only (i.e. it can, but does not have to, include other plant tissue, organs or cells). Similarly, a “leaf sample” refers to a sample that comprises leaf tissue—it does not necessarily, but can, consist of leaf tissue only (i.e. it can, but does not have to, include other plant tissue, organs or cells).

In one embodiment, a sample is obtained once the plant has started to emerge from the soil. By way of example, the sample may be obtained from the wild grass at any time post emergence, for example by hand collection.

Routine methods may be used to obtain the test sample from the plant. For example by crushing the plant tissue in a buffer for extracting protein or mRNA.

The level of biomarker in a sample of wild grass can be can be measured in a number of ways, including: measuring the mRNA that encodes the protein biomarker; measuring the amount of protein biomarker; or measuring the activity of the protein biomarker.

Any known mRNA detection method may be used to detect the level of mRNA (e.g. GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”)) in a sample.

For example, the level of a specific mRNA in a sample can be determined both by in situ and by in vitro formats. mRNA may be detected using Southern or Northern blot analysis, polymerase chain reaction or probe arrays. In one embodiment a sample may be contacted with a nucleic acid molecule (i.e. a probe, such as a labeled probe) that can specifically hybridize to the specific mRNA (e.g. the GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”) mRNA). The probe may be, for example, a complement to a full-length GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”) nucleic acid molecule, or a portion thereof, such as an nucleic acid molecule of at least 10, 15, 30, 50, 100, 250 or 500 nucleotides in length and which specifically hybridizes under stringent conditions to a GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”) nucleic acid molecule.

The term “hybridisation” as used herein shall include “the process by which a strand of nucleic acid joins with a complementary strand through base pairing” as well as the process of amplification as carried out in polymerase chain reaction (PCR) technologies. Hybridisation conditions are based on the melting temperature (Tm) of the nucleotide binding complex, as taught in Berger and immel (1987, Guide to Molecular Cloning Techniques, Methods in Enzymology, Vol. 152, Academic Press, San Diego Calif.), and confer a defined “stringency” as explained below. Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences. In a preferred aspect, the present invention covers the use of nucleotide sequences that can hybridise to the nucleotide sequences discussed herein, or the complement thereof, under stringent conditions (e.g. 50° C. and 0.2×SSC). In a more preferred aspect, the present invention covers the use of nucleotide sequences that can hybridise to the nucleotide sequences discussed herein, or the complement thereof, under high stringency conditions (e.g. 65° C. and 0.1×SSC).

Alternatively, the level of a specific mRNA in a sample may be evaluated with nucleic acid amplification, for example by rtPCR, ligase chain reaction, self sustained sequence replication, transcriptional amplification or any other nucleic acid amplification method, followed by the detection of the amplified molecules using techniques known in the art.

Any known protein detection method may be used to detect the level of protein (e.g. GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”) protein) in a sample.

Generally, protein detection methods comprise contacting an agent that selectively binds to a protein, for example an anti-TSJT1, an anti-OPR1, an anti-PHGDH1, an anti-GSTF2, an anti-GSTU2, an anti-NAD-dependent epimerase/dehydratase or an anti-NADPH:quinone oxidoreductase 1 antibody, with a sample to determine the level of the specific protein (i.e. TSJT1, OPR1, PHGDH1, GSTF2, GSTU2, NAD-dependent epimerase/dehydratase or NADPH:quinone oxidoreductase 1) in the sample. Preferably, the agent or antibody is labeled, for example with a detectable label. Suitable antibodies may be polyclonal or monoclonal. An antibody fragment such as a Fab or F(ab′)2 may be used.

As used herein the term “labeled”, refers to direct labeling of the probe or antibody by coupling (i.e., physically linking) a detectable substance to the probe or antibody, as well as indirect labeling of the probe or antibody by reactivity with a detectable substance.

The level of a specific protein biomarker in a sample may be determined by techniques known in the art, such as enzyme linked immunosorbent assays (ELISAs), immunoprecipitation, immunofluorescence, enzyme immunoassay (EIA), radioimmunoassay (RIA), Western blot analysis, and Lateral Flow Devices (LFDs) utilizing a membrane bound antibody specific to the protein biomarker. Alternatively, the level of a specific biomarker protein in a sample can be detected and quantified using mass spectrometry. Such methods are routine in the art.

The level of protein biomarker in a sample may also be determined by determining the level of protein biomarker activity in a sample.

Methods of the invention further comprise comparing the level or activity of the at least one biomarker in the test sample with the level or activity of the at least one biomarker in a control sample or with a predetermined reference level for the at least one biomarker.

In one embodiment, methods of the invention include contacting a control sample with a compound or agent capable of detecting a specific biomarker mRNA (e.g. TSJT1 mRNA, OPR1 mRNA, PHGDH1 mRNA, GSTF2 mRNA, GSTU2 mRNA, NAD-dependent epimerase/dehydratase mRNA or NADPH:quinone oxidoreductase 1 mRNA), and comparing the level of the biomarker mRNA in the control sample with the level of biomarker mRNA in the test sample.

In another embodiment, the methods of the invention include contacting the control sample with a compound or agent capable of detecting a specific biomarker protein (e.g. TSJT1 protein, OPR1 protein, PHGDH1 protein, GSTF2 protein, GSTU2 protein, NAD-dependent epimerase/dehydratase protein or NADPH:quinone oxidoreductase 1 protein), and comparing the level of the biomarker protein in the control sample with the presence of the biomarker protein in the test sample.

As used herein “reference level” or “control”, refers to a sample having a normal level of biomarker (e.g. TSJT1, OPR1, PHGDH1 A, GSTF2, GSTU2, NAD-dependent epimerase/dehydratase or NADPH:quinone oxidoreductase 1) expression, for example a sample obtained from a herbicide susceptible wild grass plant from the same species. The terms “herbicide sensitive” and herbicide susceptible” are used interchangeably herein. Alternatively, the reference level may be comprised of a biomarker expression level from a reference database, which may be used to generate a pre-determined cut off value, i.e. a score that is statistically predictive of NTSR herbicide resistance that is based on metabolic based resistance). In one embodiment, the predetermined reference level is the average level of the biomarker protein in a herbicide sensitive (or herbicide susceptible) wild grass of the same species.

Suitably, the control sample or reference sample is obtained using the same method as the method used to obtain a test sample (i.e. the sample is of the same part of the plant, same sample size etc). Alternatively, or in addition, the control sample or reference sample is normalized as discussed below.

Alternatively, predictions may be based on the normalized expression level of the specific biomarker. Expression levels are normalized by correcting the absolute expression level of the biomarker in a sample by comparing its expression to the expression of a reference nucleic acid that is not a marker, e.g., an mRNA or protein that is constitutively expressed. This normalization allows the comparison of the expression level in one sample to another sample, or between samples from different sources. This normalized expression can then optionally be compared to a reference standard or control.

For example, when measuring a biomarker in a stem sample the biomarker may be expressed as an absolute concentration or, alternatively, it may be normalized against a known protein constitutively expressed in stems, such as actin.

As mentioned above, the methods of the invention may involve determining the level of one biomarker in a sample, and determining the level of at least one further biomarker in the sample (or in an equivalent sample derived from the wild grass. Preferably, the level of the at least one further biomarker is determined using any one of the above mentioned methods.

The level of at least one further biomarker may be determined in the same sample or a different sample to the level of the first biomarker.

In one embodiment, the level of the biomarker of interest in the test sample is increased by at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, at least 3.9, at least 4.0, at least 4.1, at least 4.2, at least 4.3, at least 4.4, at least 4.5, at least 4.6, at least 4.7, at least 4.8, at least 4.9, at least 5.0 fold compared to the control sample or predetermined reference sample. In one embodiment, 1 the level of the at least one biomarker in the test sample is increased by at least 1.2 fold, at least 1.5 fold, at least 2 fold, at least 2.5 fold, at least 5 fold, at least 7.5 fold, at least 10 fold, at least 15 fold etc compared to the control sample or predetermined reference level.

Kits and Assay Devices

A kit for identifying NTSR in wild grass, wherein the NTSR is based upon metabolic based resistance , are provided. The kit comprises a detectably labelled agent that specifically binds to a biomarker selected from the group consisting of: GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”).

The components of the kit may be housed in a container that is suitable for transportation.

Details on the biomarkers is given above and apply equally here. Suitably, the biomarker may be protein or mRNA.

The term “detectably labelled agent” refers to a binding partner that interacts (i.e. binds) specifically with the biomarker of interest (i.e. GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) or stem-specific protein TSJT1 (“TSJT1”)) and is also capable of being detected e.g. directly (such as via a fluorescent tag) or indirectly (such as via a labelled secondary antibody). The detectably labelled agent is therefore a selective binding partner for the biomarker of interest. Selective binding partners may include antibodies that selectively bind to one of the biomarker of interest.

The kit may comprise one or more (i.e. two, three, four, five, six, seven or more) distinct detectably labelled agent(s). In one embodiment, a different biomarker selected from the group consisting of GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”) is specifically bound by each of the distinct detectably labelled agents. In other words, each of the distinct detectably labelled agents has a distinct biomarker specificity.

In some embodiments, the kits include the detectably labelled agent(s) on a continuous (e.g. solid) surface, such as a lateral flow surface. Alternatively, in embodiments comprising more than one detectably labelled agent, the detectably labelled agent(s) may be located in distinct (i.e. spatially separate) zones on a (e.g. solid) surface, such as a multiwall micro-titre plate. Other appropriate surfaces and containers that are well known in the art may also form part of the kit of the invention.

In one embodiment, the kit further comprises one or more reagents for detecting the detectably labelled agent. Suitable reagents are well known in the art and include but are not limited to standard reagents and buffers required to perform any one of the appropriate detection methods that may be used (and are well known in the art). In one embodiment, the kit comprises one or more of the following: ball bearing(s), extraction buffer, extraction bottle and a lateral flow device lateral flow device.

In addition, the kits may include instructional materials containing directions (i.e., protocols) for the use of the materials provided in the kit. While the instructional materials typically comprise written or printed materials, they may be provided in any medium capable of storing such instructions and communicating them to an end user. Suitable media include, but are not limited to, electronic storage media (e.g., magnetic discs, tapes, cartridges, chips) and optical media (e.g., CD ROM). The media may include addresses to internet sites that provide the instructional materials. Such instructions may be in accordance with any of the methods or uses detailed herein.

An assay device is also provided for identifying non-target-site herbicide resistance (NTSR) of wild grass, wherein the NTSR is based upon metabolic based resistance. The device comprises a surface with a detectably labelled agent located thereon, wherein the detectably labelled agent specifically binds to a biomarker selected from the group consisting of: GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”).

Detectably labelled agent(s) that specifically bind to the biomarker(s) of interest are described in detail elsewhere herein. As described above in the context of kits, one or more of such detectably labelled agents may be present. This applies equally to the assay devices provided herein, which may comprise one or more (i.e. two, three, four, five, six, seven or more) distinct detectably labelled agent(s). In one embodiment, a different biomarker selected from the group consisting of GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”) is specifically bound by each of the distinct detectably labelled agents. In other words, each of the distinct detectably labelled agents has a distinct biomarker specificity.

The assay device comprises a surface upon which the detectably labelled agents are located. Appropriate surfaces include a continuous (e.g. solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device of the invention.

Assay devices are also provided comprising at least two detectably labelled agents located on the surface, wherein the detectably labelled agents specifically bind to different biomarkers selected from the group consisting of GSTU2, D-3-phosphoglycerate dehydrogenase 1 (“PHGDH1”), 12-oxophytodienoate reductase 1 (“OPR1”), GSTF2, NADPH:quinone oxidoreductase 1 (“NQO1”), NAD-dependent epimerase/dehydratase (“NDE/D”) and stem-specific protein TSJT1 (“TSJT1”). Appropriate surfaces are detailed above. The at least two detectably labelled agents may be located in distinct (i.e. spatially separate) zones on a (e.g. solid) surface, such as a multiwell micro-titre plate.

Appropriate assay device formats therefore include but are not limited to device formats suitable for performing any one of lateral flow, dot blot, ELISA, or surface plasmon resonance assays for identifying NTSR in wild grass, wherein the NTSR is based on metabolic based resistance (i.e. by detecting the presence, level or absence of the biomarker of interest).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

### EXAMPLES

Adaptation of plant resistance to herbicides is an example of a rapid evolution in the face of human-influenced environmental change. To explain the relatively rapid occurrence of NTSR, it is proposed to be based on a subset of pre-existing stress responses to abiotic stresses that are naturally present in weed populations, and have been selected for adaption towards herbicide resistance (Legator et al., 2014). However, empirical data testing this theory is lacking, due to two main areas of gap in knowledge: 1) a fundamental understanding of the functional molecular pathways that give rise to NTSR, 2) characterisation of pre-existing stress responses in herbicide sensitive black-grass. Several of the enzyme classes that have been characterised so far in NTSR, such as GSTs, and P450s are also involved in abiotic stress. Whole transcriptomics studies have additionally indicated the involvement of several other molecular processes in weed species (including black-grass), ranging from gene and protein regulation through to stress and defence response (Gardin et al., 2015). These whole transcriptomics analyses (typically capturing thousands of differentially expressed genes between herbicide resistant and susceptible plants) indicate that NTSR is a highly quantitative trait, indicating multiple components of the plant's immune system that is adapted towards a rapid herbicide resistance. However, a comparison in response of specific enzymes in both NTSR and stress response of herbicide susceptible weeds has not been reported.

The inventors have used a functional genomic (proteomics) led approach to characterise differences in protein expression in non-target site herbicide resistant black-grass populations compared to herbicide susceptible (HS) plants. The NTSR proteome was then compared with changes in protein abundance in black-grass evoked by classic biotic and abiotic stresses relevant to field conditions including exposure to a herbicide safener used in post-emergence weed control. The HS population was compared to several NTSR populations consisting of: i) “real field” populations, considered to have undergone selection for NTSR by exposure to a combination of multiple herbicides in different geographical areas, ii) two experimental populations (originating from a HS population), each selected for NTSR by exposure to single active ingredients (either pendimethalin or fenoxaprop). Using real field populations allowed the inventors to identify realistic variability in NTSR gene expression, whilst in the experimental populations the background genetic variation between unrelated black-grass populations was reduced. In addition, the populations derived from the repeated exposure to single classes of herbicide with completely different modes of action, explored the potential for NTSR to evolve through different mechanisms. These studies demonstrated that NTSR may be achieved by multiple genetic routes depending on the type of herbicide exposure. Additionally, whilst little evidence was found to support the hypothesis that herbicide resistance is based on pre-existing stress responses, novel NTSR linked proteins strengthen the link between herbicide resistance in plants and drug resistance in mammalian tumour cells.

Black-Grass NTSR Proteome Exhibits Population Variability

Field Sourced Populations

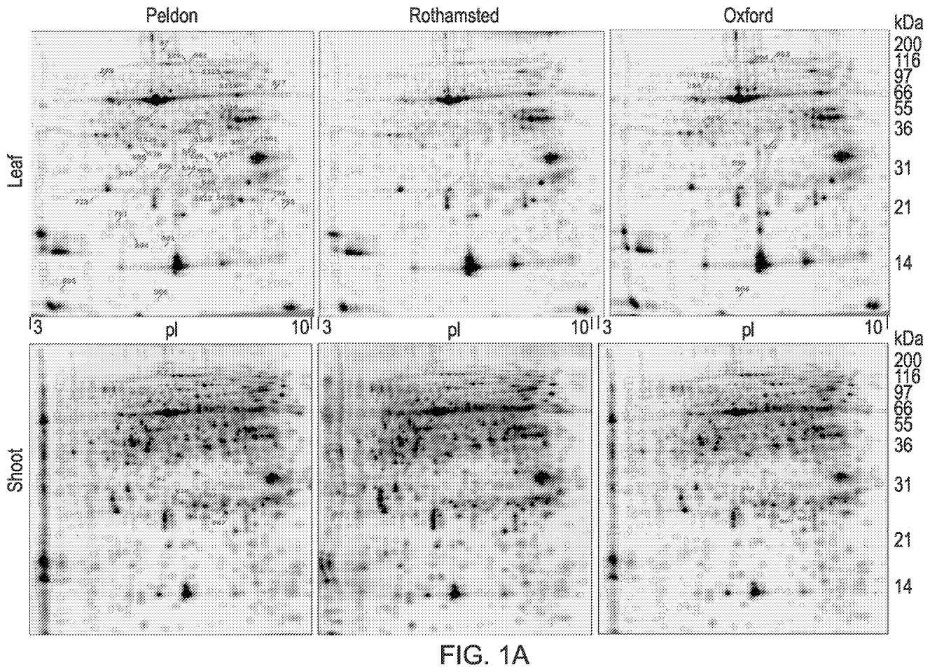

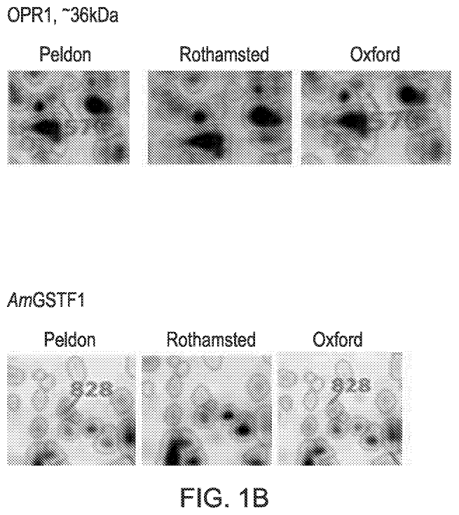

The inventors used a proteomics approach (DiGE) to compare functional differences in Black-grass gene expression between a herbicide susceptible (HS) population with two populations collected from different wheat field sites (in Oxford and Essex) that are known to exhibit resistance against multiple herbicides via non-target site resistance (NTSR). In total 894, and 1037 protein spots were detected within leaf and shoot tissue consecutively (FIG. 1). The smaller number in leaf tissue is likely owing to dominance of photosynthesis proteins, most notably Rubisco Large and Small Subunits at around 60 kDa and 14 kDa.

At the leaf level, 62 protein spots had a 1.5 to 15 fold change in protein synthesis in MHR populations compared to the HS population. Of those with an increased abundance (43 protein spots), the majority (30) were specifically upregulated in Peldon, five specifically in Oxford and only eight protein spots common to both populations. The analysis of the plant stem tissue was considered to be of greater interest than the leaf tissue, as the stem organ contains the meristematic cells targeted by the majority of graminicides used in wild-grass control. In the black-grass stems a greater number of spots were detected (1037), with fewer spots enhanced in the two NTSR populations (24 proteins) compared to HS. Furthermore, there was greater similarity in the protein spots up-regulated in the two NTSR populations; of the 20 protein spots upregulated, the majority (13) were commonly enhanced in both NTSR populations compared to HS. These included several proteins previously linked to NTSR; GSTs (AmGSTF1(2d), AmGSTF1(2c), GSTU2, GSTF2), OPR1 through transcriptome analysis (Table 1). The most highly synthesised of these in both Oxford and Peldon was a D-3-phosphoglycerate dehydrogenase (PHGDH). PHGDH is an essential for the first step of the non-photosynthetic (phosphorylated) pathway of L-serine biosynthesis, a conserved route to the synthesis of cysteine and glycine in eukaryotes. Interestingly, over synthesis of an analogue of PHGDH has also been found to be associated with drug resistance in human tumour cells. Although plants have multiple pathways to synthesise serine, recent studies have shown that phosphorylated pathway that PHGDH participates in has an essential role in serine biosynthesis for developmental signalling (Possemato et al., 2011).

Experimentally Selected Populations

The studies with field-derived resistant populations demonstrated that the respective proteomes could differ in independent NTSR populations. However, given that the two populations have no known relationship to the HS population, some of these differences could have arisen due to background genetic variation not linked to herbicide resistance. To test this, the inventors extended the proteomic comparison to two additional populations originating from the HS Rothamsted population that had undergone 5-7 years of experimentally controlled selection under a single herbicide regime, either Fenoxaprop or Pendimethalin (Supplementary Methods S1). These herbicides were used because whereas resistance to fenoxaprop can arise through both TSR- and NTSR-based mechanisms (Cocker et al., 1999), evolved tolerance to pendimethalin in black-grass is most commonly associated with enhanced detoxification (James et al., 1995). The inventors then tested these populations for cross-resistance to other herbicides at either full or ¾ field rate (Supplementary Methods S2). Despite being selected under exposure to a single herbicidal mode of action (inhibition of microtubule assembly), the Pendimethalin population was also resistant against several herbicides with ACCase mode of action (fenoxaprop, clodinafop, cycloxidim), but not ALS or cell division MOA (FIG. 18). PCR of the respective ACCases in the selected population (Marshall et al., 2013), confirmed that resistance was not due to TSR-related mechanisms. In contrast, at the application rates tested, the population isolated following repeated selection with fenoxaprop was only resistant toward the selecting herbicide, showing no cross-resistance to other ACCase-inhibitors, or the other classes of herbicides tested. PCR of the ACCases present in the resistant plants showed no evidence of TSR, suggesting that a fenoxaprop-specific NTSR mechanism had been selected for; a phenomenon previously determined in field populations exposed to this herbicide (Cocker et al., 1999). From these experiments, it was concluded that the pendimethalin- and fenoxaprop-selected populations were exhibiting different sub-classes of NTSR and that both were distinct from the more comprehensive herbicide cross-resistance determined in the Peldon and Oxford field-sourced resistant populations (Marshall et al., 2013).

The results with the two ‘forced-resistance’ populations showed surprising differences both between each other and with the field-selected populations. The pendimethalin-selected line, that showed NTSR toward all three ACCase inhibitors (fenoxaprop, clodinafop, and cycloxydim), but not toward the herbicides targeting ALS or cell division, shared a common 10 upregulated protein spots with those also enhanced in the field collected NTSR populations. Of these ten, the most similarly upregulated in the Peldon and Oxford plants were AmGSTF1, AmGSTF2, AmGSTU2, OPR1 and PHGDH 1. In contrast, the fenoxaprop-selected line, that only showed NTSR toward this one ACCase inhibitor, had a distinct proteome profile from both the field-derived and pendimethalin-resistant populations. The proteins upregulated in the fenoxaprop line included O-acetylserine (thiol)-lyase (functioning in cysteine synthesis; S283), UDP-glycosyltransferase (UGT91C1; secondary metabolism; S282), eukaryotic translation initiation factor 3 subunit I (hormone metabolism; S64), uroporphyrinogen decarboxylase 1 (tetrapyrrole biosynthesis; S271), and triosephosphate isomerase (energy and metabolism; S110).

TABLE 1
Stem proteins that were differentially expressed in NTSR populations relative to the HS
population, but not differentially expressed in HS (also known as WTS) stressed plants.

| | | | | | | |
|---|---|---|---|---|---|---|
| | | | | Score | Theoretical | |
| Spot Id | Id Method | Protein Id | Species | (MASCOT) | mass | pH |

Photosynthesis

| | | | | | | |
|---|---|---|---|---|---|---|
| S215 | PMF | ATP synthase CF1 alpha subunit | Oryza australiensis | 170 | 123386 | 9.88 |
| S62 | PMF | hydroxyphenylpyruvate reductase | Brachypodium distachyon | 205 | 106849 | 10.14 |
| | | isoform X1 | | | | |
| S169 | PMF | glyceraldehyde-3-phosphate | Brachypodium distachyon | 485 | 60807 | 8.67 |
| | | dehydrogenase A | | | | |

Cell wall

| | | | | | | |
|---|---|---|---|---|---|---|
| S172 | PMF | UDP-D-apiose/UDP-D-xylose | Brachypodium distachyon | 426 | 43291 | 7.02 |
| | | synthase | | | | |
| S58 | PMF | UDP-glucuronic acid | Brachypodium distachyon | 244 | 75082 | 9.71 |
| | | decarboxylase 6 | | | | |
| S17 | LC-MS | Beta-D-glucan exohydrolase | Hordeum vulgare | 132 | 43190 | |
| S12 | PMF | Beta-D-glucan exohydrolase | Triticum aestivum | 214 | 43190 | 9.1 |

Lipid metabolism

| | | | | | | |
|---|---|---|---|---|---|---|
| S164 | PMF | 3-hydroxyacyl-[acyl-carrier- | Setaria italica | 150 | 46602 | 9.72 |
| | | protein] dehydratase FabZ-like | | | | |

Amino acid metabolism

| | | | | | | |
|---|---|---|---|---|---|---|
| *S27 | | D-3-phosphoglycerate | Brachypodium distachyon | 324 | 74838 | 6.88 |
| | | dehydrogenase 1 | | | | |
| S25 | | D-3-phosphoglycerate | Brachypodium distachyon | 183 | 74838 | 6.88 |
| | | dehydrogenase 1, | | | | |
| S283 | PMF | O-acetylserine (thiol)-lyase | Aegilops tauschii | 264 | 49667 | 9.98 |
| | | (cysteine synthase) | | | | |

Secondary metabolism

| | | | | | | |
|---|---|---|---|---|---|---|
| S282 | PMF | UDP-glycosyltransferase (91C1) | Brachypodium distachyon | 109 | 57124 | 5.95 |
| | | (rhamnose: rhamnosyltransferase) | | | | |

Hormone metabolism

| | | | | | | |
|---|---|---|---|---|---|---|
| S64 | PMF | eukaryotic translation | Triticum aestivum | 369 | 57445 | 9.33 |
| | | initiation factor 3 subunit i | | | | |
| *S55 | PMF | 12-oxophytodienoate reductase 1 | Triticum turgidum | 193 | 64157 | 6.03 |

Tetrapyrrole synthesis

| | | | | | | |
|---|---|---|---|---|---|---|
| S271 | PMF | Uroporphyrinogen | Brachypodium distachyon | 172 | 43944 | 8.59 |
| | | decarboxylase 1 | | | | |
| S209 | PMF | oxygen-dependent | Brachypodium distachyon | 66 | 62532 | 6.9 |
| | | coproporphyrinogen-III oxidase | | | | |

Stress and defense

| | | | | | | |
|---|---|---|---|---|---|---|
| S21 | PMF | Hsp70-Hsp90 organizing protein | Brachypodium distachyon | 94 | 85381 | 8.78 |
| S281 | PMF | Hsp70-Hsp90 organizing protein | Brachypodium distachyon | 219 | 85381 | 8.78 |
| S87 | PMF | Basic endochitinase A precursor | Triticum aestivum | 242 | 64806 | 9.74 |
| S106 | PMF | AmGSTF1 (2d) | Alopecurus myosuroides | 101 | 9034 | 8.04 |
| S105 | PMF | GSTF2 | Triticum aestivum | 78 | 29276 | |
| *S129 | PMF | GSTU2 | Brachypodium distachyon | | | |

Redox

| | | | | | | |
|---|---|---|---|---|---|---|
| S284 | PMF | L-ascorbate peroxidase 2 | Aegilops tauschii | 117 | 49142 | 6.99 |
| S219 | PMF | L-ascorbate peroxidase 7 | Brachypodium distachyon | 195 | 52917 | 9.18 |
| S203 | PMF | peroxiredoxin-2E-2, | Brachypodium distachyon | 199 | 43208 | 9.31 |
| | | chloroplastic-like | | | | |
| S163 | PMF | superoxide dismutase [Cu—Zn], | Brachypodium distachyon | 457 | 41479 | 7.75 |
| | | chloroplastic | | | | |
| S147 | PMF | Cu/Zn superoxide dismutase | Triticum aestivum | 550 | 34950 | 7.79 |
| S52 | PMF | Peroxidase 2 | Oryza sativa Japonica | 420; 74 | 57446 | 8.42 |
| S208 | PMF | 2-methylene-furan-3-one | Brachypodium distachyon | 128; 118 | 102661; 31575 | 9.32; 9.33 |
| | | reductase (quinone reductase) | | | | |

Nucleotide metabolism

| | | | | | | |
|---|---|---|---|---|---|---|
| S218 | PMF | Soluble inorganic pyrophosphatase | Brachypodium distachyon | 86 | 25744 | 9.2 |
| S133 | PMF | deoxyuridine 5′-triphosphate | Brachypodium distachyon | 439 | 20517 | 5.2 |
| | | nucleotidohydrolase | | | | |

Transcription

| | | | | | | |
|---|---|---|---|---|---|---|
| S16 | LC-MS | DEAD-box ATP-dependent RNA | Brachypodium distachyon | 145 | 100855 | |
| | | helicase 37 | | | | |
| S18 | LC-MS | probable pre-mRNA-splicing factor | | 58 | 123031 | |
| | | ATP-dependent RNA helicase | | | | |
| | | isoform X1 | | | | |
| *S101 | LC-MS | stem-specific protein TSJT1 | Brachypodium distachyon | 317 | 149653 | |
| S132 | PMF | transcription factor BTF3 | Brachypodium distachyon | 194 | 37022 | 9.4 |
| | | homolog 4 | | | | |
| S99 | PMF | Glycine-rich protein (cold shock | Triticum aestivum | 193 | 17013 | 8.72 |
| | | protein) | | | | |
| S280 | PMF | membrane-associated 30 kDa | Brachypodium distachyon | 65 | 67670 | 9.56 |
| | | protein, chloroplastic | | | | |

Protein synthesis, and degradation

| | | | | | | |
|---|---|---|---|---|---|---|
| S2 | PMF | alanine--tRNA ligase | Brachypodium distachyon | 171 | 129041 | 6.54 |
| S204 | PMF | 60S acidic ribosomal protein P3 | Aegilops tauschii | 113 | 31807 | 7.12 |
| *S102 | LC-MS | NAD-dependent epimerase/ | Zea mays | 107 | 44419 | |
| | | dehydratase | | | | |
| S6 | PMF | puromycin-sensitive | Brachypodium distachyon | 84 | 145714 | 8.72 |
| | | aminopeptidase isoform X2 | | | | |
| *S103 | PMF | NADPH: quinone oxidoreductase 1 | Brachypodium distachyon | 109 | 36406 | |
| S98 | LC-MS | NAD-dependent epimerase/ | | 503 | 44419 | |
| | | dehydratase | | | | |
| S5 | PMF | ATP-dependent Clp protease ATP- | Brachypodium distachyon | 89 | 115910 | 8.81 |
| | | binding (chaperone) subunit ClpC2 | | | | |
| S8 | PMF | ATP-dependent Clp protease ATP- | Setaria italica | 68 | 88889 | 7.36 |
| | | binding (chaperone) subunit ClpC1 | | | | |
| S196 | PMF | ATP-dependent Clp protease ATP- | Setaria italica | 193 | 88889 | 7.36 |
| | | binding (chaperone) subunit ClpC1 | | | | |
| S198 | PMF | ATP-dependent Clp protease ATP- | Setaria italica | 236 | 88889 | 7.36 |
| | | binding (chaperone) subunit ClpC1 | | | | |
| S7 | PMF | ATP-dependent Clp protease ATP- | Brachypodium distachyon | 262 | 115910 | 8.81 |
| | | binding (chaperone) subunit ClpC2 | | | | |
| S111 | PMF | 20 kDa chaperonin, chloroplastic- | Brachypodium distachyon | 335 | 46068 | 9.51 |
| | | like | | | | |

Signalling

| | | | | | |
|---|---|---|---|---|---|
| S20 | LC-MS | NAD-dependent epimerase/ | Medicago truncatula | 55 | 37375 |
| | | dehydratase family protein | | | |
| S19 | LC-MS | YTH domain-containing family | Brachypodium distachyon | 57 | 75143 |
| | | protein 1-like | | | |

Unknown

| | | | | | | |
|---|---|---|---|---|---|---|
| S73 | PMF | RNA-binding protein FUS | Setaria italica | 190 | 23248 | 6.58 |
| S202 | PMF | 23 kDa jasmonate-induced protein | Oryza sativa Japonica | 121 | 26053 | 9.34 |
| S214 | PMF | Plant UBX domain-containing | Brachypodium distachyon | 65 | 53614 | 8.82 |
| | | protein 1 | | | | |
| S212 | PMF | Probable (S)-ureidoglycine | Brachypodium distachyon | 91 | 44647 | 8.59 |
| | | aminohydrolase | | | | |

Energy and metabolism (glycolysis, electron transport)

| | | | | | | |
|---|---|---|---|---|---|---|
| S110 | PMF | triosephosphate isomerase, | Brachypodium distachyon | 420 | 36071 | 9.6 |
| | | cytosolic | | | | |
| S63 | PMF | malate dehydrogenase | Setaria italica | 77 | 52696 | 6.95 |
| S95 | PMF | Gamma carbonic anhydrase 2 | Brachypodium distachyon | 168 | 37484 | 9.33 |
| S97 | PMF | gamma carbonic anhydrase 2 | Oryza sativa Japonica | 159 | 22782 | 10.81 |
| S213 | PMF | Gamma carbonic anhydrase 1 | Brachypodium distachyon | 77 | 51881 | 8.55 |
| S96 | PMF | Ubiquinol oxidase 2 | Brachypodium distachyon | 165 | 57288 | 9.79 |

| | PMF: sequence | | | | | | |
|---|---|---|---|---|---|---|---|
| | coverage (%)/LC-MS: | Number | | | | | Transcript |
| Spot Id | number of spectra | of Peldon | Oxford | Fenoxaprop | | Pendimethalin | expression# |
| Photosynthesis | | | | | | | |
| S215 | 2% | 2 | — | — | 1.6 | 1.2 | — |
| S62 | 3% | 2 | — | — | 1.1 | 1.5 | — |
| S169 | 13% | 7 | — | — | 1.6 | 0.9 | 10.2 |
| Cell wall | | | | | | | |
| S172 | 15% | 4 | 1.1 | 1.0 | 1.5 | 0.8 | — |
| S58 | 4% | 3 | — | — | 1.5 | 0.9 | — |
| S17 | 7 | 4 | 0.8 | 0.9 | 1.1 | 1.6 | — |
| S12 | 8% | 2 | — | — | 1.2 | 1.5 | — |
| Lipid metabolism | | | | | | | |
| S164 | 6% | 2 | — | — | 1.5 | 0.8 | — |
| Amino acid metabolism | | | | | | | |
| *S27 | 9% | 4 | 4.0 | 3.4 | 1.2 | 3.2 | 5.3 |
| S25 | 4% | | 2.7 | 2.6 | 1.2 | 3.0 | 5.3 |
| S283 | 6% | 2 | — | — | 1.8 | 1.0 | 17.8 |
| Secondary metabolism | | | | | | | |
| S282 | 5% | 3 | — | — | 1.5 | 1.0 | |
| Hormone metabolism | | | | | | | |
| S64 | 9% | 3 | — | — | 2.2 | 0.6 | |
| *S55 | 4% | 2 | 2.6 | 2.5 | 1.1 | 4.1 | 101.2 |
| Tetrapyrrole synthesis | | | | | | | |
| S271 | 5% | 2 | — | — | 1.7 | 1.5 | 0.4 |
| S209 | 2% | 1 | — | — | 0.7 | 1.8 | |
| Stress and defence | | | | | | | |
| S21 | 1% | 1 | — | — | 1.5 | 1.3 | |
| S281 | 4% | 2 | — | — | 2.8 | 0.8 | |
| S87 | 6% | 2 | — | — | 0.5 | 0.5 | |
| S106 | 25% | | 2.9 | 2.5 | 1.6 | 2.1 | 26.2 |
| S105 | | | 3.7 | 3.5 | 1.1 | 2.2 | |
| *S129 | | | 6.2 | 2.2 | 1.3 | 2.0 | 141.5 |
| Redox | | | | | | | |
| S284 | 2% | 1 | | | | | |
| S219 | 5% | 2 | — | — | 2.1 | 0.7 | |
| S203 | 5% | 1 | — | — | 1.7 | 1.3 | 2.8 |
| S163 | 13% | 3 | — | — | 1.5 | 0.8 | 4.7 |
| S147 | 18% | 3 | — | — | 0.3 | 0.5 | 4.7 |
| S52 | 12% | 4 | — | — | 1.5 | 0.8 | |
| S208 | 1%; 6% | 2 | — | — | 0.7 | 1.8 | |
| Nucleotide metabolism | | | | | | | |
| S218 | 5% | 1 | — | — | 1.9 | 1.3 | 9.6 |
| S133 | 26% | 3 | — | — | 0.5 | 0.7 | |
| Transcription | | | | | | | |
| S16 | 5 | 4 | 0.8 | 0.9 | 1.1 | 1.6 | |
| S18 | 2 | 2 | 0.8 | 0.9 | 1.1 | 1.6 | |
| *S101 | 10 | 5 | 3.1 | 4.0 | 1.1 | 1.7 | |
| S132 | 6% | 1 | — | — | 0.3 | 2.3 | |
| S99 | 11% | 1 | — | — | 0.9 | 1.7 | |
| S280 | 1% | 1 | — | — | 1.1 | 2.7 | |
| Protein synthesis, and degradation | | | | | | | |
| S2 | 2% | 2 | — | — | 0.5 | 1.0 | |
| S204 | 5% | 1 | — | — | 1.7 | 1.3 | |
| *S102 | 7 | 4 | 3.1 | 4.0 | 1.1 | 1.7 | |
| S6 | 1% | 1 | — | — | 1.8 | 1.0 | |
| *S103 | 4% | 1 | 3.1 | 4.0 | 1.1 | 1.7 | 46.7 |
| S98 | 20 | 6 | 3.3 | 1.0 | 1.3 | 0.7 | |
| S5 | 2% | 1 | — | — | 1.8 | 1.0 | 0.3 |
| S8 | 1% | 1 | — | — | 1.8 | 1.0 | 0.3 |
| S196 | 4% | 2 | — | — | 0.4 | 1.0 | 0.3 |
| S198 | 4% | 2 | — | — | 0.5 | 1.0 | 0.3 |
| S7 | 4% | 3 | — | — | 1.8 | 1.6 | 0.3 |
| S111 | 11% | 3 | — | — | 2.4 | 0.9 | |
| Signalling | | | | | | | |
| S20 | 2 | 2 | 0.8 | 0.9 | 1.1 | 1.6 | |
| S19 | 2 | 2 | 0.8 | 0.9 | 1.1 | 1.6 | |
| Unknown | | | | | | | |
| S73 | 11% | 2 | — | — | 0.4 | 0.7 | |
| S202 | 6% | 2 | — | — | 1.3 | 0.6 | |
| S214 | 2% | 1 | — | — | 1.5 | 1.7 | |
| S212 | 3% | 1 | — | — | 1.5 | 1.7 | |
| Energy and metabolism (glycolysis, electron transport) | | | | | | | |
| S110 | 15% | 4 | — | — | 2.4 | 0.9 | 14.2 |
| S63 | 3% | 2 | — | — | 1.1 | 1.5 | 0.6 |
| S95 | 8% | 2 | — | — | 0.9 | 2.8 | |
| S97 | 12% | 2 | — | — | 0.9 | 2.8 | |
| S213 | 2% | 1 | — | — | 1.5 | 1.7 | |
| S96 | 4% | 2 | — | — | 0.9 | 2.8 | |

*Asterisks indicate seven proteins that are commonly up-regulated in NTSR populations, showing potential for use as diagnostic biomarkers.
#Fold change of transcripts that also exhibited differential gene expression (in Peldon relative to the HS population) corresponding to proteins.

Similar to the field populations, the inventors detected around 1100 protein spots in the stem tissue, 69 of which were differentially expressed between NTSR and HS populations. Comparing these differentially expressed spots with the differentially expressed field collected NTSR populations (103 in total), only 1 (AmGSTF1) had similar expression (up-regulated) in all four NTSR populations (FIG. 2). This confirms that AmGSTF1 plays a fundamental role in NTSR based herbicide resistance (Cummins et al., 1999; Cummins et al., 2013), with the detection of this protein being the basis of a current LFD diagnostic in development with MoLogic. The Pendimethalin selected line shared an additional 10 upregulated protein spots with the field collected populations, including the xenome linked GSTs and OPR1 and the PHGDH. However, a high proportion of protein spots exhibited specific up/down regulation in the Pendimethalin and particularly in the Fenoxaprop populations compared to HS, indicating a high degree of narrow range selection towards herbicide specific tolerance linked proteins (FIG. 18).

NTSR in Black-Grass is Distinct From the Response to Herbicide Safeners and Commonly Observed Biotic and Abiotic Stress Responses

Chemical safeners are co-applied with herbicides to specifically enhance herbicide tolerance and hence selectivity in cereals. Previous studies have shown that proteins linked to NTSR in black-grass can be induced in HS plants exposed to chemical safeners (Cummins et al., 2009). However, the mechanisms by which safening is induced in wild grasses is unknown, and any common relationships with the evolution of NTSR inferred rather than determined. In this study, exposure of HS black-grass to a wheat safener (cloquintocet-mexyl) resulted in major change in the HS proteome, with 25% of the resolved polypeptides in leaves (131 of 512 protein spots), and 3% of the stem proteome (26 of 874 protein spots) showing changes in abundance relative to controls.

Given the differences between NTSR and the response to safeners, the inventors extended the proteomic comparison to include abiotic and biotic stresses that black-grass would naturally be exposed to under field conditions. Stress responses included a range of long term (nitrogen deficiency) through to short-term (wound) stresses, and had visible effects on plant biomass. Whilst testing for evidence of a link between NTSR and naturally evolved stress responses, this allowed us to observe whether responses to the safener exhibited similarity to other stress responses in the HS population. These results indicated that black-grass stress induced changes in protein expression were similar to safener induced changes but distinct from those determined in NTSR plants. Importantly, the comparison of NTSR associated proteins and HS stress associated proteins highlighted 8 NTSR biomarkers that are not induced in HS plants exposed to either safeners or stresses (FIG. 3). The first 7 NTSR biomarkers are previously unknown, and the 8th NTSR biomarker has been previously published and is included for reference.

Black-Grass NTSR Proteome Enables Functional Validation of Differential Gene and Metabolite Expression

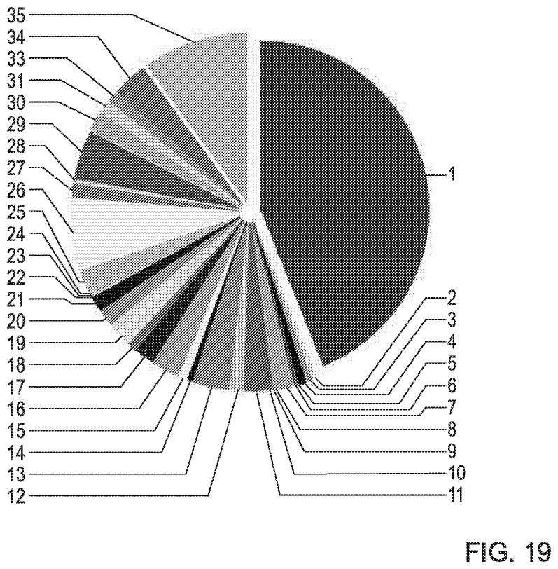

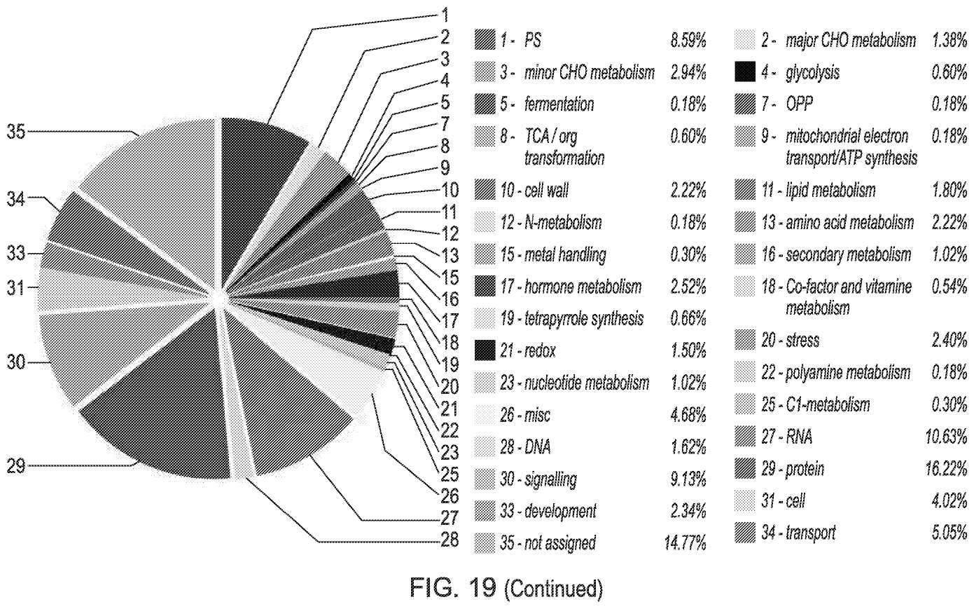

To validate links between differential protein synthesis and gene expression, the inventors created a Black-Grass Next Generation Sequencing (NGS) database, and a metabolomics database comparing a Multiple Herbicide Resistant (Peldon) and the HS population. Similar to the proteomics comparison, the whole transcriptomics identified differentially expressed genes covering multiple pathways, indicating the complexity of NTSR (FIG. 19).

Comparing the inventor's transcriptome and proteome databases, the inventors identified several genes and metabolites matching the inventor's proteins that have previously not been linked to NTSR (Table 1). A notable example is that of Vitamin B6 synthesis (pyridoxal biosynthesis protein PDX1.1-like) which increased in NTSR at both transcript and protein level, as well as a glycosylated pyridoxal metabolite which increased in the NTSR population (5′-O-beta-D-Glucosylpyridoxine) (Table 2). Glycosylation is a mechanism of reversible metabolite deactivation, which may stimulate its continued production and thus accumulation. Vitamin B6 is purported to have two roles of potential relevance in NTSR, 1) antioxidant; 2) enzyme cofactor, notably in the transulfuration pathway of glutathione biosynthesis. Interestingly, the transulfuration pathway is linked to two other pathways that had several genes and proteins upregulated in NTSR, serine and anthocyanin biosynthesis (Table 2). Serine is involved at an early stage in the transulfuration pathway, reacting with homocysteine to form cystathionine with the enzyme cystathione B-synthase and cofactor pyroxidal-5-phosphate (vitamin B6), which leads to production of cysteine required for glutathione biosynthesis. Glutathione is well characterised for its role in detoxification and vacuolar sequestration of heterocyclic organic anions (Edwards and Dixon 2000). This bears a very striking resemblance to the last stage of anthocyanin biosynthesis, glutathionylation of cyanidin glucosides, that enables anthocyanins to be transported to the vacuole via a GS-X (glutathione) pump (Goodman et al., 2004).

TABLE 2
Pathways linked to the increased synthesis of metabolites in NTSR
(Peldon) populations compared to HS

| | | | |
|---|---|---|---|
| | | Fold | |
| Code | Identification | change | Reference |

Serine biosynthesis

| | | | |
|---|---|---|---|
| Protein | S25 | D-3-phosphoglycerate | 2.7 |
| | | dehydrogenase | |
| Transcript | | D-3-phosphoglycerate | 5.3 |
| | | dehydrogenase | |

Glutathione biosynthesis (transulfuration pathway)

| | | | |
|---|---|---|---|
| Gene | | Pyridoxal biosynthesis protein | 10.0 |
| | | PDX1.1-like | |
| | | Pyridoxal biosynthesis protein | |
| Protein | L100 | PDX1.1-like | 1.9 |
| Metabolite | | 5′-O-beta-D-Glucosylpyridoxine | 5.4 |

Anthocyanin biosynthesis

| | | | | |
|---|---|---|---|---|
| Transcript | R00052282 | 4-coumarate--CoA ligase 3, 4CL 3 | 2.2 | |
| | | (4-coumaroyl-CoA synthase 3) | | |
| | | Chalcone--flavonone isomerase, CHI | | |
| Transcript | R00028278 | (Chalcone isomerase) | 2.0 | |
| | | Dihydroflavonol-4-reductase, DFR | | |
| Transcript | R00002631 | (Dihydrokaempferol 4-reductase) | 2.4 | |
| Transcript | R00014499 | Anthocyanidin 5,3-O- | 5.3 | |
| | | glucosyltransferase, AGT (UDP- | | |
| | | glucose: anthocyanidin 5,3-O- | | |
| | | glucosyltransferase) | | |
| Protein | S282 | Anthocyanin-UGT | 1.5 | |
| Transcript | R00053652 | Anthocyanin 5-aromatic | 5.3 | |
| | | Acyltransferase (5AT) | | |
| Metabolite | | 1-o-feruloyl glucose | 5.1 | (Matsuba et |
| | | | | al., 2010) |

The inventors observed transcriptomic and proteomic similarities between the NTSR field populations (oxford and peldon) and the NTSR experimentally selected pendimethalin population, which were not in common with the single herbicide resistant experimentally selected fenoxaprop population. In particular, seven novel biomarkers were identified by the inventors as being upregulated at both the transcriptome and proteome level in the three black-grass populations displaying NTSR (arising from NTSR based upon metabolic based resistance), which were not upregulated in the fenoxaprop single herbicide resistant population (see FIG. 3).

Discussion

Proteomic Characterisation of Multiple Herbicide Resistance (NTSR)

This is the first reported study to identify and measure the production of proteins in weeds associated with non-target site herbicide resistance (NTSR), a damaging trait enabling weeds to detoxify multiple classes of chemical control agents. The inventors demonstrate that the expression of specific proteins in differently derived NTSR populations is both qualitatively and quantitatively different. Classically, NTSR has been attributed to multiple genes contributing to the xenome, which are involved in the detoxification and transport of herbicide metabolites into plant vacuoles (Cummins et al., 2013). In agreement, the inventors found several enhanced Glutathione S-transferases (U2, F1, F2), as well as 12-oxophytodienoate reductase 1 (OPR1), an enzyme closely related to oxidoreductases linked to the detoxification of explosives in plants and microbes (Mezzari et al., 2005). However, the majority of polypeptides detected have not previously been reported to be linked to NTSR, or herbicide tolerance in plants.

Several previous studies have shown changes in gene expression associated with NTSR in wild grasses, including black-grass (Gardin et al., 2015) and annual Rye-grass (Gaines et al., 2014). Whilst these studies have identified differences between herbicide resistant and susceptible weed populations at the transcriptional level, regulation of translation can prevent many transcripts from having a functional role, whilst post-translational modification can alter the functional annotation of transcripts. To the inventor's knowledge, this is the first report of changes in the proteome of a wild grass linked to the evolution of NTSR, with previous protein expression studies linked to herbicide tolerance linked to the effects of safeners in wheat species (Zhang and Riechers, 2004). Moreover, the majority of studies characterise herbicide resistance associated genes in a single population. In this study, the inventors show that NTSR associated protein expression can differ between populations, potentially due to different types of herbicide exposure indicated by the populations experimentally selected towards either Fenoxaprop or Pendimethalin. It is known that selection by different herbicide classes can induce different sets of proteins in green algae, including glutathione S-transferases (Nestler et al, 2012).

In addition to xenome detoxification pathways, changes in the proteome in NTSR black-grass showed functional perturbation in the pathways involved in protein synthesis, sulphur assimilation, reduced cofactor synthesis and turnover, as well as redox and oxidative stress responses. The differential synthesis of several of these previously uncharacterised NTSR associated proteins were supported by differential gene expression at the transcriptomic level and metabolite synthesis. This related to both direct matches, but also indicated pathways involved in the transulfuration pathway relating to glutathione biosynthesis. Although the role of Glutathione S-transferases (GSTs and glutathione) in xenobiotic detoxification is well known, the molecular components contributing to their increased synthesis in NTSR are poorly understood. This study indicates that several enzymes such as D-3-phosphoglycerate dehydrogenase (PHGDH, serine biosynthesis) and pyridoxal biosynthesis protein (PDX1.1-like, vitamin B6) may crucial roles in GST mediated non target site resistance.

Multiple Routes to NTSR

In this study, the inventors analysed the proteome of populations collected from field sites, as well as populations that had been experimentally selected for resistance against one of two herbicides. Interestingly the Fenoxaprop selected line did not exhibit resistance towards any other herbicide, indicating specificity in its resistance type, and moreover, specificity in the proteins that were overproduced in this population alone. Furthermore, the expression of AmGSTF1 in the fenoxaprop population indicates that AmGSTF1 alone does not confer multiple herbicide NTSR, but merely confers some level of NTSR—however this study indicates that NTSR can be specific to herbicide types, depending on what the plant has been exposed to (adapted resistance towards).

The ability to focus on the small number of proteins consistently associated with NTSR and to then study their variation in content in the different resistant populations, provided new insight into the potential mechanisms of this type of herbicide resistance. Effectively three types of NTSR could be identified from the field and forced-selection studies with each associated with changes in the respective proteomes. Firstly, a comprehensive NTSR was identified in the field-derived Peldon and Oxford populations that was associated with multiple resistance to both ALS and ACCase active herbicides. The inventors have previously termed this Multiple Herbicide Resistance (MHR), as it is independent of herbicide chemistry and mode of action (Cummins et al., 2013). Then there was the group-specific class of NTSR determined in the pendimethalin-selected plants that only conferred cross-resistance to different ACCase active herbicides. Finally, a compound-specific type of NTSR that conferred resistance to only a single herbicide, as selected by fenoxaprop, was identified.

The three different types of NTSR (MHR, group & compound specific), were each associated with characteristic changes in their proteomes. In terms of identifying those proteins associated with the different types of NTSR, only AmGSTF1 was found to be up-regulated in all three classes of resistance. This further confirmed the importance of this protein as a core component of NTSR in black-grass (Cummins et al., 1999; Cummins et al., 2013). Similarly, orthologs of AmGSTF1 have also been shown to be upregulated in NTSR in other wild grasses, such as L. rigidum (Cummins et al., 2013). While the fenoxaprop-specific NTSR clearly exemplified the potential for unique evolutionary routes to NTSR, the changes in the respective proteome gave no immediate clues as to the resistance mechanisms in play.

In contrast, the proteins upregulated in both the group-specific and MHR black-grass did give some insight into NTSR mechanisms. Four of the 8 proteins, identified were xenome components, suggesting a strong functional link of NTSR to detoxification. In addition to AmGSTF1, a further phi-class protein termed AmGSTF2 and a tau enzyme AmGSTU2 were identified. The latter was of interest, as it was by far the most abundant GST in the MHR plants. To date, it has not proven possible to predict plant GST function from sequence alone, but the elevation of a group of these proteins in NTSR plants, linked to a coordinated elevation in glutathione content (Cummins et al., 2013), suggests they have complimentary functions in protecting the plants from herbicide injury. Another xenome protein of interest was OPR1. The OPRs are flavin mononucleotide-binding enzymes that are classically associated with the conversion of 12-oxo-cis-10,15-phytodienoate to 3-oxo(-cis-2′-pentenyl) cyclopentane-1-octanoate, a key step in jasmonic acid biosynthesis (Stintzi and Browse, 2000). The OPRs can be subdivided into 3 classes, with the OPR1 enhanced in the NTSR plants, associated with the reductive detoxification of xenobiotics such as the explosive TNT, rather than jasmonate synthesis (Beynon et al., 2009).

Similarity Between NTSR in Weeds and in Tumour Cells

Using the global proteomics approach, several polypeptides which were enhanced in NTSR black-grass were shown to be orthologous to proteins linked to multiple drug resistance (MDR) in human tumours. Previous studies had shown that AmGSTF1 in NTSR black-grass was functionally similar to the evolutionarily distinct HsGSTP1 in MDR tumours in human (Cummins et al., 2013). In the current study further direct evidence of parallel evolution of resistance mechanisms was determined. D-3-phosphoglycerate dehydrogenase (PHGDH) was found to be enhanced in NTSR black-grass and has also been linked to certain breast cancers, where its overexpression is required for cell proliferation (Possemato et al., 2011). As such, PHDH is a target for chemotherapy using protein inhibitors. PHGDH, using NAD+/NADH as a cofactor catalyses the transition of 3-phosphoglycerate into 3-phosphohydroxypyruvate, the first and rate-limiting step in the phosphorylated pathway of serine biosynthesis. Interestingly, clinical evaluation of a PHGDH inhibitor demonstrated that only tumour cells that overproduce PHGDH were sensitive to its knock-down (Possemato et al., 2011). The exact mechanism of PHGDH participation in either cancer development or herbicide resistance remains to be discovered, though several hypotheses have been proposed. In PHGDH knocked-out cancer cells, rather than having an alteration in intracellular serine levels, there is a drop in α-ketoglutarate, another output of the pathway. It was recently reported that PHGDH converts α-ketoglutarate to d-2-hydroxyglutarate (d-2HG, an oncometabolite), which is further converted to 2-oxoglutarate, which is sensitive to oxidative stress in plants and known to be elevated in plants after exposure to oxidative stress (Lehmann et al., 2012). This indicates that the over synthesis of 2-oxoglutarate may in effect be “primed” towards a repeated oxidative stress. However, PHGDH related α-ketoglutarate may in addition play a direct role in herbicide tolerance; several bacteria have been reported to degrade phenoxyalkanoic herbicides using the α-ketoglutarate-dependent 2,4-dichlorophenoxyacetate dioxygenase (Gazitua et al., 2010). In tumour cells, it has been proposed that over synthesis of PHGDH is linked to the ability to import serine from its extracellular environment has been perturbed, leading to its dependent on elevated do novo synthesis of serine via PHGDH.

Similarly, NADH-cytochrome b5 reductase (CBR), enhanced two-fold in NTSR (Peldon) leaf tissue, has also been linked to drug metabolism in human cancer cells, where it has a 3-7 fold increase in enzyme activity (Barham et at 1996). CBRs are a family of flavoproteins that catalyse the reduction of coenzyme Q and cytochrome b5 using NADH as an electron donor in a one-electron transfer reaction. This protein is required for several metabolic functions including desaturation and elongation of fatty acids and is linked to xenobiotic metabolism in both plant and mammalian systems, as well as correct reproductive functioning in plants (Wayne et al 2013). Studies have indicated its potential to activate bioreductive drugs in the treatment of cancer (Barham et al 1996). Interestingly, CBRs may have a functional overlap with P450 reductases, more commonly associated with xenobiotic detoxification (Wayne et al. 2013).

Demonstration of Use of Additional Biomarkers in Identifying NTSR in Black-Grass

Proteomics data have identified a set of eight proteins that are specifically up-regulated in non-target site resistance populations of Alopecurus myosuroides (Am) compared with herbicide susceptible plants.

To expand the proteomic data we further investigated the expression of five up-regulated proteins by Western blotting. Rabbit polyclonal antibodies were raised against pair of peptides for each of the following proteins: Am12-oxophytodienoate-reductase, AmD-3-phosphoglycerate dehydrogenase, AmGstu2, AmGstF2 and AmNADPH-quinone oxidoreductase 1.

To optimize the specificity of the antibodies towards a broad range of species of weeds and crops, pairs of peptides were selected based on conserved regions in multi sequence alignment of homologues for each of the five selected proteins. This would allow contrasting the expression of the same cohort of proteins across a number of related weeds and also follow the expression of these proteins in crops (monocot) after treatment with herbicide safeners. The selected pair of peptides for each protein is described in Table 4.

TABLE 4
Sequence of the peptides used to raise antisera.

| | |
|---|---|
| 12-oxophytodienoate- | 1) 14-mer CTSDPVVGYTDYPFL |
| reductase | 2) 15-mer CEIPGIVDDFRKAARN |
| D-3-phosphoglycerate | 1) 13-mer CRQVDQPGMIGSV |
| dehydrogenase | 2) 14-mer CGEIPAIEEFVFLKL |
| GstF2 | 1) 12-mer CLEEAGVEYELVP |
| | 2) 11-mer CAMVDVWLEVEA |
| GstU2 | 1) 10-mer CSAVHSGIKIF |
| | 2) 12-mer CSKGQYFGGESV |
| NAPH-quinone 1 | 1) 20-mer |
| oxidoreductase | CEESIPGLQIDHVDISDLPL |
| | 2) 20-mer |
| | VRAFDDPPKFDAAGNLTHAC |

Table 4: The extra cysteine added to allow conjugation of the peptides to the carrier and not part of the native sequence is shown in bold.

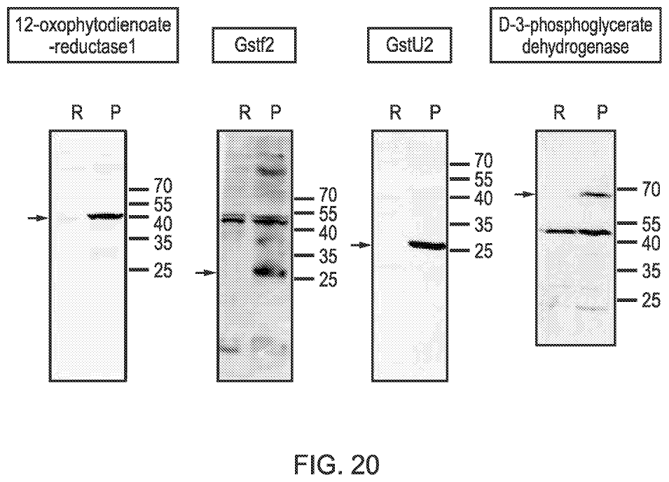

The generated antibodies were tested by Western Blots against protein extracts from Black grass stem from Rothamsted, which is an herbicide susceptible population, and Peldon which has a high incidence of non-target site resistance (FIG. 20). In each of the four tested antisera (the antisera for AmNADPH-quinone oxidoreductase 1 are in production) the Western blot detected proteins with the expected apparent relative molecular mass (Mr): Am12-oxophytodienoate-reductase (˜40 kDa), AmD-3-phosphoglycerate dehydrogenase (˜65 kDa), AmGstu2 (˜26 kDa) AmGstF2 (˜26 kDa). Consistent with the proteomics data the proteins detected by Western blot all were up-regulated in the Peldon samples, demonstrating an enhanced expression of those proteins in herbicide resistant populations. These data further confirm the utility of these proteins as biomarkers of non-target site resistance in black-grass and their potential for development for applications in other weeds.

Conclusions

In this study, the inventors found little evidence for the hypothesis that resistance towards multiple herbicides is either induced by safeners, or is based on pre-existing stress responses. The proteomics led identification of functional genes (biomarkers) associated with herbicide resistance has several potential applications arising from this work including i) new targets for disruption of the NTSR phenotype through the use of classical agrochemistry or gene/protein disruption technology; ii) genes that can be used to engineer herbicide tolerance into crop plants; iii) development of diagnostics for distinct classes of NTSR (demonstrating varying susceptibilities to graminicides) based on differential protein biomarker expression. The outcomes of this study are of greatest relevance to NTSR in black-grass, but is highly likely to be of relevance to herbicide resistance in related wild grasses.

Materials and Methods

Black-Grass Populations

A black-grass population originating from Rothamsted Broadbalk experimental grounds (guaranteed herbicide free) was used as the standard Herbicide Susceptible (HS) population, collected in 2009. This was used as a standard control for two “real” NTSR populations originating from wheat field sites in Essex (Peldon) and Oxford and two experimental NTSR populations that had undergone directed selection to two types of herbicidal active ingredients (Fenoxaprop and Pendimethalin). Both experimental populations originated from the Rothamsted HS population in 2004. Further details on the selection regime to create the distinct Fenoxaprop and Pendimethalin resistant populations are provided in Supplementary Methods S1/S2.

All plant populations were germinated and grown for 2.5 weeks (to 2 tiller stage) in compost (John Innes No.2). Plants were then transferred to 10 cm pots (8 plants per pot) containing autoclaved sharp sand mixed with 25% perlite, and watered with full strength Hoaglands solution. Growth conditions were set to a light:dark cycle of 16:8 with temperature of 21° C:15° C. Plant tissues (leaf, shoot, root) were harvested at 5.5 weeks old (6 tiller stage), weighed for fresh biomass, snap frozen in liquid nitrogen and stored at −80° C.

Stress Treatments

To test whether NTSR associated constitutive proteins are stress inducible (/exploring the hypothesis that NTSR is based on pre-existing stress responses), the inventors designed a multi-stress experiment consisting of nine stress treatments that black-grass could naturally be exposed to in the field. This consisted of i) biotic stresses: plant growth promoting rhizobacteria (PGPR), insect (aphid—Sitobion avenae) abiotic (wound, heat, drought, salt, nitrogen deficiency) and a commonly used wheat safener (cloquintocet-mexyl). The stress conditions were applied at time-points consistent with their nature to ensure a proteomic response at harvest. Nitrogen deficiency (10% N Hoaglands recipe), salt stress (building from 40 mM to 160 mM NaCl diluted in Hoaglands) and rhizobacteria (10 ml Pseudomonas aeruginosa 7NSK2 inoculum per plant, prepared as previously described (Tétard-Jones et al., 2007)) were applied three weeks before harvest. Two weeks before harvest, the insect stress (two adult “english grain” aphids, Sitobion avenae) were placed on each plant and insect proof bags fitted to all experimental plants. Aphids were allowed to naturally reproduce, reaching an average population size of 281 individuals at harvest. The drought (osmotic) stress was artificially controlled, by application of Polyethylene Glycol (PEG) 8000 over 11 days prior to harvest, starting with an osmotic pressure of −0.15 MPa building up to −0.66 MPa as previously described (Michel, 1983). The safener (30 mM cloquintocet-mexyl) was watered onto plants 4 days before harvest. The heat stress was applied as a gradual ramp from 21° C. to 40° C. in 3° C. increments over 6 hours, and maintained at 40° C. for a further 9 hours before harvest. The gradual heat ramp was intended to simulate a natural rising temperature, acclimating the plants to heat stress, to induce a heat response without protein degradation that can be caused by sudden heat shock (Altschuler and Mascarenhas, 1982). Wounding stress was applied by pressing a sewing wheel along the length of each plant leaf 9 hours prior to harvest.

Differential Gel Electrophoresis (DiGE)

Leaf and shoot proteomes were extracted and analysed following 2-D Fluorescence Difference Gel Electrophoresis (DiGE) techniques according to (Tétard-Jones et al., 2013). In summary, proteins were extracted from 600mg tissue using TCA/acetone precipitation, purified (Clean-Up kit) and quantified using standard kits (GE Healthcare). For each protein sample, 50 ug was labelled with minimal CyDyes, including an internal standard composed of an aliquot of each sample. Resulting scanned gel images were analysed using Progenesis SameSpots software (Non Linear Dynamics). Overall, 800-1100 distinct protein spots were matched across the DiGE gels and used in statistical analyses. Protein spots that had a significant change in their volume between black-grass populations and stress treatments (p<0.05 and/or fold change>1.5) were selected for protein identification by Peptide Mass Fingerprinting by Bioscience Technology Facility at University of York.

Transcriptomics and Metabolomics

NTSR (Peldon) and HS (Rothamsted) seed lines of black-grass were planted into 12 cm diameter terracotta pots in a peat based compost using 20 seeds per pot. Plants (3 biological replicates) were grown under controlled glasshouse conditions at the University of York and The Food and Environment Research Agency (Fera). After 3 weeks of growth plants were harvested directly into liquid nitrogen, cutting from just above the soil line and stored at −80° C. for less than 2 weeks prior to transcriptomics and metabolomics analysis as detailed in the Supplementary Methods S3 and S4.

Supplementary Methods S1

Treatment programme used to experimentally select for Fenoxaprop resistance starting with a Wild Type Sensitive population (HS population) that had not previously been exposed to herbicides. The program started with the Rothamsted HS population in 1990. Variations in doses used for each round of selection reflect changes in yearly environmental conditions.

TABLE 3

| | | | Nos. plants | | |
|---|---|---|---|---|---|
| Selection | Year | Nos. plants | grown on for | | Selecting dose |
| year | done | treated | seed production | Selection % | used |
| 1^{st} | 1990 | 456 | 10 | 2.2% | 70 g fenoxaprop |
| | | | | | racemate/ha |
| | | | | | (=×0.58 field rate) |
| 2^{nd} | 1991 | 506 | 3 | 0.6% | 100 + 100 g |
| | | | | | fenoxaprop |
| | | | | | racemate/ha |
| | | | | | (=×1.67 field rate) |
| 3^{rd} | 1991/92 | 524 | 15 | 2.9% | 125 g fenoxaprop |
| | | | | | racemate/ha |
| | | | | | (=×1.04 field rate) |
| 4^{th} | 1997/98 | 486 | 43 | 8.8% | 27.5 g fenoxaprop |
| containers | | | | | isomer/ha |
| | | | | | (×0.5 field rate) |
| 5^{th} | 1998/99 | 50 | 8 | 16% | 220 g fenoxaprop |
| | | | | | isomer/ha |
| | | | | | (×4 field rate) |
| 6^{th} | 2014/15 | 26 | 26 | 100% | 18 plants × 55 g |
| | | | | All survived | fenoxaprop |
| | | | | | isomer/ha + |
| | | | | | 8 plants × 110 g |
| | | | | | fenoxaprop |
| | | | | | isomer/ha |

Table 3 Notes: 1) Other doses were used in the series of selection trials but the above relate specifically to the OUT population. The doses varied because the level of control varied between trials to some degree, so the populations receiving the dose that gave the ‘best’ degree of selection tended to be used for growing on to produce seed. For example, control in outdoor containers in 1997/98 was particularly high due to favourable conditions for herbicide activity, so plants surviving half the field rate were grown on as few plants survived full rate. 2) The plants from the final selection were using for proteomic characterisation and additionally evaluated for cross-resistance patterns to other herbicides (FIG. 18).

Supplementary Methods S2

Cross resistance test of Pendimethalin and Fenoxaprop selected lines (WTS and HS are used interchangeably).

| Populations (4): | WTS ROTH04 (baseline for Pendimethalin population) | |
|---|---|---|
| | WTS ROTH09 | |
| | ROTH PEND 14 (8 yrs selection) | |
| | ROTH FEN OUT (6 yrs selection) | |
| Treatments (9): | 1 Untreated | |
| | 2 Fenoxaprop* | 0.75 l/ha |
| | 3 Clodinafop* | 0.0938 l/ha + 0.5% vol. Toil |
| | 4 Cycloxydim* | 0.563 l/ha + 0.5% vol. Toil |
| | 5 Mesosulfuron + | 300 l/ha + 0.5% vol. BioPower |
| | iodosulfuron* | |
| | 6 Sulfometuron* | 99.75 g/ha |
| | 7 Pyroxsulam* | 250 g/ha + 1.0% Biosyl |
| | 8 Pendimethalin# | 1200 g ai/ha |
| | 9 Flufenacet# | 240 g ai/ha |
| * 3/4 field rate | | |
| | # field rate | |

| | |
|---|---|
| Reps (3): | 3 (6 plants/pot) |
| Total plants/pots: | 108 pots in total |
| | 27 pots/population, 5 dishes/population |

Supplementary Methods S3

IonTorrent Transcriptome Sequencing and Assembly

RNA was extracted from frozen plant shoot tissue using RNeasy Mini Kit (Qiagen) according to the manufacturer's protocols. Total RNA (15-20 ug) was DNase treated (Turbo DNA-free; Ambion) and then concentrated using the RNA Clean & Concentrate kit (Zymo Research). mRNA was purified from the total RNA using two passages through the Oligotex mRNA Mini kit (Qiagen) and quality and profiles were assessed on a 2200TapeStation Nucleic Acids System (Agilent Technologies). RNA-Seq libraries were prepared from each mRNA sample (approx 25-50 ng) using the Ion Total RNA-Seq kit v2 (Life Technologies), with an RNase III treatment time of 2.5-3 min. Yields and library sizes were assessed using the 2200 TapeStation Nucleic Acids System (Agilent Technologies). Diluted library aliquots were combined in pairs in equimolar amounts and used for template preparation using the Ion OneTouch 200 Template Kit v2 DL (Life Technologies), prior to loading onto a 318 chip and sequenced on an Ion Torrent PGM prepared as per the manufacturer's instructions (IonPGM200Kit; Life Technologies).

De novo assembly of sequenced mRNA libraries was performed by Fios Genomics (www). Assembly of the black-grass BAM files were converted to SAM files and then FASTQ format files with samtools (Li et al., 2009) and SamToFastq (v1.9; Picard tools). The FASTQC versions of the files were then assessed by Fastqc version 0.10.1 (FastQC) and reads trimmed to remove the first 10 bases at the 5′ end. The 3′ end of the reads were also trimmed wherever the quality scores dropped below 20. Any reads shorter than 40 nt were excluded from further analysis. rRNA and tRNA species from Oryza sativa (Rice) were used as a proxy for the same molecules in black grass. These sequences were taken from the genome and gff files downloaded as part of IRSGP (build 4). Fasta sequences representing the molecules in the gff files were extracted using bedtools (Quinlan and Hall, 2010), the RNA-Seq reads were matched using bowtie2 using Ion-torrent specific settings: (local alignments, very-sensitive) as described by Langmead and Salzberg, 2012a). Those reads that did not match the rRNA species were considered in the later stages of the assessment.

Assembly

MIRA (Chevreux et al., 2004) was utilised for the assembly, which is considered to be the most reliable algorithm in instances where the assembly is generated from Ion-Torrent and not guided by a reference sequence, i.e. de novo transcriptome assembly (Loman et al., 2012; Rothberg et al., 2011). The trimmed, rRNA filtered, read files from runs of the second replicate of either population were concatenated and used in two individual, accurate, EST specific assemblies. The reads that were left unassembled in either case, and reads from the unused runs (first and third replicate of both populations) that failed to map to the generated contigs from the same run (using bowtie2 as previously described), were combined. These additional collections of reads were used in two additional MIRA accurate EST assemblies. Reads, from either assembly, that had no alignment to another read were discarded.

Generation of the Unigene Sets

The assembled contigs from both black-grass populations were combined and clustered into representative sequences using the CD-HIT-EST (Li and Godzik, 2006). All sequences with a gapped alignment of 95% over a minimum 90% of the shorter reads length were clustered. One set was generated for each population, and a final set was generated from the combined contigs of both populations.

Annotation & Super-Contig Assembly (Scaffolding by Association)

Blast databases were generated from each of the following sources: NCBI's species-specific unigenenucleic acid databanks (for Oryza Sativa, Hordeum vulgare and Arabidopsis thaliana), KOG (coreprotein set), uniprot (protein) and finally the PlantCyc enzyme set (protein). Function of the contigs was assigned by blasting each contig against each database in turn using either blastx, for protein databases, or blastn, for nucleic acid based databases. Only matches where an E-value of less than 1×10⁻⁵ was returned were considered valid hits. The results were post processed so that contigs were assembled into super-contigs (sets of contigs where the order and direction of each contig relative to the others can be inferred, but gaps between them exist) according to the alignment along the species specific transcripts. As this relies on a level of homology between the species, and the species for which full length transcripts are available, this process may not be required, however it was completed to aid understanding of the assembly data.

ORF Prediction

The prediction of open-reading frames (ORFs) in the transcripts was achieved using Trinity's longest ORF prediction method (Parra et al., 2000).

Differential Expression

The reads of each subject were aligned to the final combined contig set using bowtie2 optimising the search for local-alignments, and using the very-accurate approach (Langmead and Salzberg, 2012b). The number of reads from each sample that aligned to a given contig were determined using custom scripts. These values were then summed across all contigs that matched to the same uniprot definition; the counts were adjusted downwards to reflect the occasions when a read aligned to different contigs of the same uniprot reference thereby preventing the double-counting of each read. The differential expression analysis was performed within the R statistics environment v2.15 (R Core Team, 2013) using the EBSeq (Leng et al., 2013). Multiple testing statics (FDR and FWER) were applied via the multtest package (Pollard et al., 2005). Differential expression is considered from the respect of herbicide resistant population relative to the susceptible population, therefore up-regulation refers to the increased number of alignments in the resistant individuals rather than the sensitive individuals.

Gene Ontology Analysis

The GO-codes for each uniprot identifier were assigned using the biomaRt package (Durinck et al., 2009), using Uniprot's unimart interface (Magrane and Consortium, 2011). Enrichment analysis was performed using the hypergeometric calculation within R (Team, 2013) where enrichment in gene sets that had a p-value less than 0.05 and showed either a 2-fold increase or decrease in the posterior calculations were considered significant.

Data Analysis

Transcriptome (contig) sequences that were significantly differently expressed between the susceptible and resistant populations (fold change>2, FDR<0.05) were submitted to Mercator annotation using standard settings to assign functional BINs to contig sequences (Götz et al., 2008). This enabled visualisation of functions represented by sequences throughout the transcriptome that were significantly up or down regulated in the resistant population relative to the susceptible population. Given that the number of contigs mapped in the resistant population was greater overall for all 36 BINS, the inventors applied a normalisation to show the percentage of up-regulated relative to mapped contigs for each population.

Supplementary Methods S4

Metabolomics Extraction and Analysis

Extraction Method for Metabolomic Analysis

Each frozen plant was lyophilised overnight and ground into a fine powder using an A 11 basic analytical mill (IKA, Staufen, Germany). 5 mg±0.1 mg of ground sample was accurately weighed into a labelled 2 mL eppendorf tube. To 5 mg of sample, 1 ml of extraction solvent (1:1 (v/v) methanol: water) was added. Metabolites were extracted into the solvent by shaking for 30 minutes. The solid material was then removed by centrifugation at 14,000 rpm for 10 minutes at ambient temperature. To prepare samples for profiling analysis by Liquid Chromatography-High Resolution Mass Spectrometry (LC-HRMS) the supernatant was diluted 9:1 with 1:1 (v/v) methanol: water. An analytical quality control (QC) sample was created by pooling 1 ml from each final sample extract. To prepare samples for profiling analysis by 1H nuclear magnetic resonance (NMR) spectroscopy 900 μL of supernatant was taken and the methanol removed by passing a stream of nitrogen over the sample for approximately 1 hour. The remainder of the extract was lyophilised overnight. The dried sample was constituted in 700 μL of NMR sample buffer (250 mM potassium phosphate, pH=7.0; and 0.5 mM Trimethylsilyl propanoic acid, TSP, dissolved in D2O), centrifuged and 540 μL transferred to a labelled NMR tube. To the labelled NMR tube, a 60 μL aliquot of 10 mM sodium azide in D2O, (to prevent microbial growth) was added before NMR analysis.

LC-HRMS Profiling Conditions—Metabolomics

LC analysis was performed on an Accela 1250 High Speed LC system from Thermo Fisher Scientific (Waltham, Mass., USA). The analytical column used was an ACE Excel AQ (Advanced Chromatography Technologies, UK) 150 mm×3 mm, 100 Å. Mobile phase A (MPA) was 0.1% formic acid in HPLC water, mobile phase B (MPB) was 0.1% formic acid in acetonitrile. A linear gradient elution was applied over 10 minutes from 100% MPA to 100% MPB. The gradient was then held for 2 minutes at 100% MPB before re-equilibration with 100% MPA for a further 2 minutes. The LC flow rate was 0.4 mL min⁻¹ and the column temperature was 30° C. Sample injection volume was 5 μL. The MS used was an Orbitrap Velos Pro hybrid ion trap high resolution mass spectrometer (Thermo Fisher Scientific, Waltham, Mass., USA) with a mass resolution setting of 60,000 at m/z 200. Maximum injection time was 50 ms. Ionisation was by heated electrospray (HESI) with extracts analysed in both positive and negative mode. The source heater temperature was set to 450° C. with sheath gas set to 51 and aux gas at 16 (au, arbitrary units). The capillary temperature was 370° C. Sample analysis order was randomised using www.random.org and a pooled QC sample was injected every 6 injections to monitor system performance.

LC-HRMS Confirmatory Conditions—Metabolomics

After profiling a number of confirmatory analyses on the sample extracts were analysed on the Oribtrap Velos Pro using the MS/MS and MSn capabilities of the instrument. A precursor list of potentially significant accurate masses were generated from the profiling analysis and used as precursor ions for MS/MS and MSn experiments in both positive and negative ionisation modes. Collision Induced Dissociation (CID) fragmentation was employed using 35 ev. A fragmentation event was only triggered if the precursor mass had a signal of 3000 (au) or greater. FTMS/MS detection was used in order to obtain accurate mass product ions. An MS³ event was subsequently triggered on the 3 most abundant product ions of the precursor mass if the product ions had a signal of 500 (au) or greater. MS³ ions were detected by the ion trap producing nominal mass product ions. A corresponding analytical standard was analysed when available to concurrently confirm compound identity. Where an analytical standard could not be obtained, compounds were “affirmatively” identified (or dismissed) using theoretical fragmentation from Mass Frontier software (HighChem Ltd, Bratislava, Slovakia). All analytical standards were purchased from Sigma-Aldrich (Gillingham, UK).

NMR Conditions and Data Processing—Metabolomics

Spectra were acquired using a Bruker 500 MHz NMR spectrometer equipped with a 5 mm TCI cryoprobe. All spectra were acquired and processed using Topspin 2.13 patch level 6 (Bruker, Germany). Spectra were acquired using a simple single pulse 1H NMR experiment with pre-saturation. The ¹H NMR spectra were acquired at a central frequency of 500.1317700 MHz and at 300 K over a sweepwidth of 19.9947 ppm giving an acquisition time of 1.64 s using a total relaxation delay of 6.5 s per transient. 256 transient and 8 dummy scans were collected giving a total experiment time of approximately one hour. A calibrated 90° pulse length of 9.42 μs was used. Chemical shifts were referenced to TSP at 0 ppm. NMR spectra were binned using an adaptive binning algorithm to reduce the dataset size and to compensate for any minor changes in chemical shift between spectra. A wavelet coefficient of 3 was used and this resulted in data bins of varying frequency widths associated with NMR resonances. Statistical analysis was performed using a custom written graphical user interface (GUI) for Matlab (The Mathworks, Natick, Mass., USA. Version 7.4.0.287 [R2007a]) known as Metabolab.

Profiling Data Interpretation and Statistical Differentiation Analysis—LC-HRMS Data—Metabolomics

Xcalibur™ qual software, Sieve 2.1 software (both from Thermo Fisher Scientific, Waltham, Mass., USA) and Progenesis CoMet (NonLinear Dynamics, Newcastle, UK.) were used to assess the profile data. CoMet allows the upload of thermo.raw data files and performs peak picking, feature alignment, deconvolution of compounds according to potential adducts found and statistical analysis between sample groups. The most significant potential compounds (or masses), and their relative abundance, between lines can then be discovered. In this experiment peak picking was performed with a signal threshold 3000 (peak area) within the peak picking algorithm and samples were aligned against a QC sample. After peak picking and deconvolution all m/z's found were screened against an in house library of metabolites to aid in tentative compound identification using CoMet's MetaScope library searching tool. Potential significant masses between sample lines were discovered by filtering on P-value<0.01 (or in a second pass P≤0.05) after one way ANOVA and mean response fold differences between the lines 2 (both calculated by the CoMet software). Potential significant m/z's that had not been identified earlier using the MetaScope function were searched against the Metlin library (Scripps Centre For Metabolomics, California, USA) which contains information for 243,000 metabolites. Compound confirmation/affirmation of potential significant compounds were obtained using MS/MS information (see above).

### REFERENCES

Altschuler, M. and Mascarenhas, J. P. (1982) Heat shock proteins and effects of heat shock in plants. Plant Mol Biol 1, 103-115.
Barham H M, Inglis R, Chinje E C, Stratford I J. Development and validation of a spectrophotometric assay for measuring the activity of NADH: cytochrome b5 reductase in human tumour cells. Br J Cancer. 1996 October; 74(8):1188-93.
Beynon, E. R., Symons, Z. C., Jackson, R. G., Lorenz, A., Rylott, E. L. and Bruce (2009) The role of oxophytodienoate reductases in the detoxification of the explosive 2,4,6-trinitrotoluene by Arabidopsis. Plant Physiol., 151, 253-261.
Cocker, K. M., Moss, S. R. and Coleman, J. O. D. (1999) Multiple mechanisms of resistance to fenoxaprop-P-ethyl in United Kingdom and other European populations of herbicide-resistant Alopecurus myosuroides (Black-Grass). Pestic. Biochem. Physiol., 65, 169-180.
Cummins, I., Cole, D. J. and Edwards, R. (1999) A role for glutathione transferases functioning as glutathione peroxidases in resistance to multiple herbicides in black-grass. Plant J., 18, 285-292.
Cummins I, Wortley D J, Sabbadin F, He Z, Coxon C R, Straker H E, Sellars J D, Knight K, Edwards L, Hughes D, Kaundun S S, Hutchings S J, Steel P G, Edwards R., 2013. Key role for a glutathione transferase in multiple-herbicide resistance in grass weeds. Proc Natl Acad Sci U S A., 110(15):5812-7.
Gaines T A, Lorentz L, Figge A, Herrmann J, Maiwald F, Ott M C, Han H, Busi R, Yu Q, Powles S B, Beffa R., 2014. RNA-Seq transcriptome analysis to identify genes involved in metabolism-based diclofop resistance in Lolium rigidum. Plant J., 78(5):865-76.
Gardin J A, Gouzy J, Carrère S, Délye C., 2015. ALOMYbase, a resource to investigate non-target-site-based resistance to herbicides inhibiting acetolactate-synthase (ALS) in the major grass weed Alopecurus myosuroides (black-grass). BMC Genomics. 12; 16:590.
Gazitua, M. C., Slater, A. W., Melo, F. and Gonzalez, B. (2010) Novel alpha-ketoglutarate dioxygenase tfdA-related genes are found in soil DNA after exposure to phenoxyalkanoic herbicides. Environ Microbiol 12, 2411-2425.
Goodman, C. D., Casati, P. and Walbot, V. (2004) A Multidrug Resistance—Associated Protein Involved in Anthocyanin Transport in Zea mays. The Plant Cell 16, 1812-1826.
Gray J P, Karandrea S, Burgos D Z, Jaiswal A A, Heart E A., 2016. NAD(P)H-dependent quinone oxidoreductase 1 (NQO1) and cytochrome P450 oxidoreductase (CYP450OR) differentially regulate menadione-mediated alterations in redox status, survival and metabolism in pancreatic β-cells. Toxicol Lett. 2016 Nov 16; 262:1-11.
Hall, L. M., Moss, S. R. and Powles, S. B. (1997) Mechanisms of Resistance to Aryloxyphenoxypropionate Herbicides in Two Resistant Biotypes of Alopecurus myosuroides (blackgrass): Herbicide Metabolism as a Cross-Resistance Mechanism. Pesticide Biochemistry and Physiology 57, 87-98.
Holger Nestler, Ksenia J. Groh, René Schönenberger, Rik I. L. Eggen, Marc J.-F. Suter, Linking proteome responses with physiological and biochemical effects in herbicide-exposed Chlamydomonas reinhardtii, Journal of Proteomics, Volume 75, Issue 17, 2012, Pages 5370-5385.
James, E. H., Kemp, M. S. and Moss, S. R. (1995) Phytotoxicity of trifluoromethyl- and methyl-substituted dinitroaniline herbicides on resistant and susceptible populations of black-grass (Alopecurus myosuroides). Pestic. Sci., 43, 273-277.
Marshall, R., Hanley, S. J., Hull, R. & Moss, S. R. (2013) The presence of two different target-site resistance mechanisms in individual plants of Alopecurus myosuroides Huds., identified using a quick molecular test for the characterisation of six ALS and seven ACCase SNPs. Pest Man. Sci., 69, 727-737.
Lagator M, Colegrave N, Neve P., 2014. Selection history and epistatic interactions impact dynamics of adaptation to novel environmental stresses. Proc Biol Sci., 281(1794):20141679.
Lehmann, M., Laxa, M., Sweetlove, L. J., Fernie, A. R. and Obata, T. (2012) Metabolic recovery of Arabidopsis thaliana roots following cessation of oxidative stress. Metabolomics 8, 143-153.
Matsuba, Y., Sasaki, N., Tera, M., Okamura, M., Abe, Y., Okamoto, E., Nakamura, H., Funabashi, H., Takatsu, M., Saito, M., Matsuoka, H., Nagasawa, K. and Ozeki, Y. (2010) A Novel Glucosylation Reaction on Anthocyanins Catalyzed by Acyl-Glucose-Dependent Glucosyltransferase in the Petals of Carnation and Delphinium. The Plant Cell 22, 3374-3389.
Mezzari M P, Walters K, Jelinkova M, Shih M C, Just C L, Schnoor J L., 2005. Gene expression and microscopic analysis of Arabidopsis exposed to chloroacetanilide herbicides and explosive compounds. A phytoremediation approach. Plant Physiol., 138(2):858-69.
Michel, B. E. (1983) Evaluation of the Water Potentials of Solutions of Polyethylene Glycol 8000 Both in the Absence and Presence of Other Solutes. Plant Physiology 72, 66-70.
Moss, S. R. (1990) Herbicide Cross-Resistance in Slender Foxtail (Alopecurus myosuroides). Weed Science 38, 492-496.
Panozzo, S., Scarabel, L., Collavo, A., Sattin, M. Protocols for Robust Herbicide Resistance Testing in Different Weed Species. J. Vis. Exp. (101), e52923, doi:10.3791/52923 (2015)
Possemato, R., Marks, K. M., Shaul, Y. D., Pacold, M. E., Kim, D., Birsoy, K., Sethumadhavan, S., Woo, H.-K., Jang, H. G., Jha, A. K., Chen, W. W., Barrett, F. G., Stransky, N., Tsun, Z.-Y., Cowley, G. S., Barretina, J., Kalaany, N. Y., Hsu, P. P., Ottina, K., Chan, A. M., Yuan, B., Garraway, L. A., Root, D. E., Mino-Kenudson, M., Brachtel, E. F., Driggers, E. M. and Sabatini, D. M. (2011) Functional genomics reveal that the serine synthesis pathway is essential in breast cancer. Nature 476, 346-350.
Sobajima, H., Takeda, M., Sugimori, M., Kobashi, N., Kiribuchi, K., Cho, E. M., Akimoto, C., Yamaguchi, T., Minami, E., Shibuya, N., Schaller, F., Weiler, E. W., Yoshihara, T., Nishida, H., Nojiri, H., Omori, T., Nishiyama, M., Yamane, H., 2003. Cloning and characterization of a jasmonic acid-responsive gene encoding 12-oxophytodienoic acid reductase in suspension-cultured rice cells. Planta,
Stintzi, A. and Browse, J. (2000) The Arabidopsis male-sterile mutant, opr3, lacks the 12-oxophytodienoic acid reductase required for jasmonate synthesis. Proc. Natl. Acad. Sci. USA., 97, 10625-10630.
Tétard-Jones, C., Kertesz, M. A., Gallois, P. and Preziosi, R. F. (2007) Genotype-by-genotype interactions modified by a third species in a plant-insect system. Am Nat 170, 492-499.
Tétard-Jones, C., Shotton, P. N., Rempelos, L., Cooper, J., Eyre, M., Orr, C. H., Leifert, C. and Gatehouse, A. M. R. (2013) Quantitative proteomics to study the response of wheat to contrasting fertilisation regimes. Molecular Breeding 31, 379-393.
Villalba, J. M. et al. Role of cytochrome b5 reductase on the antioxidant function of coenzyme Q in the plasma membrane. Mol. Aspects Med. 18 Suppl, S7-S13 (1997).
Wayne L L1, Wallis J G, Kumar R, Markham J E, Browse J. Plant Cell. 2013 August; 25(8):3052-66. doi: 10.1105/tpc.113.113324. Epub 2013 Aug. 30. Cytochrome b5 reductase encoded by CBR1 is essential for a functional male gametophyte in Arabidopsis
Xu, X. L., Chen, S., Salinas, N. D., Tolia, N. H., Grant G. A., 2015. Comparison of Type 1 D-3-phosphoglycerate dehydrogenases reveals unique regulation in pathogenic Mycobacteria, Archives of Biochemistry and Biophysics, 570 (32-39).
Chevreux, B., Pfisterer, T., Drescher, B., Driesel, A. J., Muller, W. E., Wetter, T. and Suhai, S. (2004) Using the miraEST assembler for reliable and automated mRNA transcript assembly and SNP detection in sequenced ESTs. Genome research 14, 1147-1159.
Cingolani, P., Platts, A., Wang le, L., Coon, M., Nguyen, T., Wang, L., Land, S. J., Lu, X. and Ruden, D. M. (2012) A program for annotating and predicting the effects of single nucleotide polymorphisms, SnpEff: SNPs in the genome of Drosophila melanogaster strain w1118; iso-2; iso-3. Fly 6, 80-92.
Danecek, P., Auton, A., Abecasis, G., Albers, C. A., Banks, E., DePristo, M. A., Handsaker, R. E., Lunter, G., Marth, G. T., Sherry, S. T., McVean, G. and Durbin, R. (2011) The variant call format and VCFtools. Bioinformatics (Oxford, England) 27, 2156-2158.
Durinck, S., Spellman, P. T., Birney, E. and Huber, W. (2009) Mapping identifiers for the integration of genomic datasets with the R/Bioconductor package biomaRt. Nature protocols 4, 1184-1191.
Langmead, B. and Salzberg, S. L. (2012a) Fast gapped-read alignment with Bowtie 2. Nat Meth 9, 357-359.
Langmead, B. and Salzberg, S. L. (2012b) Fast gapped-read alignment with Bowtie 2. Nature methods 9, 357-359.
Leng, N., Dawson, J. A., Thomson, J. A., Ruotti, V., Rissman, A. I., Smits, B. M., Haag, J. D., Gould, M. N., Stewart, R. M. and Kendziorski, C. (2013) EBSeq: an empirical Bayes hierarchical model for inference in RNA-seq experiments. Bioinformatics (Oxford, England) 29, 1035-1043.
Li, H., Handsaker, B., Wysoker, A., Fennell, T., Ruan, J., Homer, N., Marth, G., Abecasis, G. and Durbin, R. (2009) The Sequence Alignment/Map format and SAMtools. Bioinformatics (Oxford, England) 25, 2078-2079.
Li, W. and Godzik, A. (2006) Cd-hit: a fast program for clustering and comparing large sets of protein or nucleotide sequences. Bioinformatics (Oxford, England) 22, 1658-1659.
Loman, N. J., Misra, R. V., Dallman, T. J., Constantinidou, C., Gharbia, S. E., Wain, J. and Pallen, M. J. (2012) Performance comparison of benchtop high-throughput sequencing platforms. Nat Biotech 30, 434-439.
Magrane, M. and Consortium, U. (2011) UniProt Knowledgebase: a hub of integrated protein data. Database: the journal of biological databases and curation 2011, bar009.
Parra, G., Blanco, E. and Guigo, R. (2000) GeneID in Drosophila. Genome research 10, 511-515.
Pollard, K. S., Dudoit, S. and van der Laan, M. J. (2005) Multiple Testing Procedures: the multtest Package and Applications to Genomics. In: Bioinformatics and Computational Biology Solutions Using R and Bioconductor (Gentleman, R., Carey, V., Huber, W., Irizarry, R. and Dudoit, S. eds), pp. 249-271. Springer New York.
Quinlan, A. R. and Hall, I. M. (2010) BEDTools: a flexible suite of utilities for comparing genomic features. Bioinformatics (Oxford, England) 26, 841-842.
Rothberg, J. M., Hinz, W., Rearick, T. M., Schultz, J., Mileski, W., Davey, M., Leamon, J. H., Johnson, K., Milgrew, M. J., Edwards, M., Hoon, J., Simons, J. F., Marran, D., Myers, J. W., Davidson, J. F., Branting, A., Nobile, J. R., Puc, B. P., Light, D., Clark, T. A., Huber, M., Branciforte, J. T., Stoner, I. B., Cawley, S. E., Lyons, M., Fu, Y., Homer, N., Sedova, M., Miao, X., Reed, B., Sabina, J., Feierstein, E., Schorn, M., Alanjary, M., Dimalanta, E., Dressman, D., Kasinskas, R., Sokolsky, T., Fidanza, J. A., Namsaraev, E., McKernan, K. J., Williams, A., Roth, G. T. and Bustillo, J. (2011) An integrated semiconductor device enabling non-optical genome sequencing. Nature 475, 348-352.
Team, R. C. (2013) R: A Language and Environment for Statistical Computing. Vienna: R Foundation for Statistical Computing.
Thiel, T., Michalek, W., Varshney, R. K. and Graner, A. (2003) Exploiting EST databases for the development and characterization of gene-derived SSR-markers in barley (Hordeum vulgare L.). TAG. Theoretical and applied genetics. Theoretische und angewandte Genetik 106, 411-422.
Matsuba, Y., Sasaki, N., Tera, M., Okamura, M., Abe, Y., Okamoto, E., Nakamura, H., Funabashi, H., Takatsu, M., Saito, M., Matsuoka, H., Nagasawa, K. and Ozeki, Y. (2010) A Novel Glucosylation Reaction on Anthocyanins Catalyzed by Acyl-Glucose-Dependent Glucosyltransferase in the Petals of Carnation and Delphinium. The Plant Cell 22, 3374-3389.

1. A method for detecting non-target site herbicide resistance (NTSR) in wild grass, comprising using at least one of:
i. GSTU2;
ii. D-3-phosphoglycerate dehydrogenase 1;
iii. 12-oxophytodienoate reductase 1;
iv. GSTF2;
v. NADPH:quinone oxidoreductase 1;
vi. NAD-dependent epimerase/dehydratase; or
vii. stem-specific protein TSJT1;
wherein the NTSR is based upon metabolic based resistance.

2. The method of claim 1, wherein the biomarker is protein or mRNA.

3. The method of claim 1, wherein the wild grass is selected from a group consisting of black grass, rye grass, wild oat and bent grass.

4. A method of identifying non-target-site herbicide resistance in wild grass, the method comprising:
i) determining the level of at least one biomarker selected from a group consisting of: GSTU2, D-3-phosphoglycerate dehydrogenase 1, 12-oxophytodienoate reductase 1, GSTF2, NADPH: quinone oxidoreductase 1, NAD-dependent epimerase/dehydratase and stem-specific protein TSJT1 in a test sample of the wild grass; and
ii) comparing the level of the at least one biomarker in the test sample with the level of the at least one biomarker in a control sample or with a predetermined reference level for the at least one biomarker;
wherein an increased level of the at least one biomarker in the test sample compared to the control sample or compared to the predetermined reference level is indicative of non-target-site herbicide resistance based upon metabolic based resistance.

5. The method of claim 4, wherein the biomarker is protein or mRNA.

6. The method of claim 4, wherein the wild grass is selected from a group consisting of black grass, rye grass, wild oat and bent grass.

7. The method of claims 4, wherein the test sample is a stem sample or a leaf sample.

8. The method of claim 4, wherein the test sample is obtained post emergence.

9. The method of claim 4, wherein the control sample is obtained from a herbicide sensitive wild grass of the same species, or the predetermined reference level is the average level of the at least one biomarker in a herbicide sensitive wild grass of the same species.

10. The method of claim 4, wherein the level of the at least one biomarker in the test sample is increased by at least 1.5 fold, at least 2 fold, at least 2.5 fold, or at least 5 fold compared to the control sample or predetermined reference level.

11. A kit for identifying non-target-site herbicide resistance (NTSR) in wild grass, wherein the NTSR is based upon metabolic based resistance, the kit comprising: a detectably labelled agent that specifically binds to a biomarker selected from a group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1.

12. The kit of claim 11, wherein the biomarker is protein or mRNA.

13. The kit of claim 11, further comprising one or more reagents for detecting the detectably labelled agent.

14. An assay device for identifying non-target-site herbicide resistance (NTSR) of wild grass, wherein the NTSR is based upon metabolic based resistance, the device comprising: a surface with a detectably labelled agent located thereon, wherein the detectably labelled agent specifically binds to a biomarker selected from a group consisting of: GSTU2, D-3-phosphoglycerate dehydrogenase 1, 12-oxophytodienoate reductase 1, GSTF2, NADPH: quinone oxidoreductase 1, NAD-dependent epimerase/dehydratase and stem-specific protein TSJT1.

15. The assay device of claim 14, comprising at least two detectably labelled agents located on the surface, wherein the detectably labelled agents specifically bind to different biomarkers selected from a group consisting of: GSTU2, D-3-phosphoglycerate dehydrogenase 1, 12-oxophytodienoate reductase 1, GSTF2, NADPH:quinone oxidoreductase 1, NAD-dependent epimerase/dehydratase and stem-specific protein TSJT1.

16. The assay device of claim 15, wherein the at least two detectably labeled agents are located in separate zones on the surface.

17. The assay device of claims 14, wherein the biomarker is protein or mRNA.

18. The assay device of claim 15, wherein the biomarker is protein or mRNA.

19. The assay device of claim 16, wherein the biomarker is protein or mRNA.
